(19) United States
(12) Patent Application Publication
(10) Pub. No.: US 2025/0006333 A1
(43) Pub. Date: Jan. 2, 2025

(54) Methods For Determining a Risk Of Medication Overuse Headache and Alerting a User of Medication Overuse Headache Risk

(71) Applicant: CURELATOR, INC, Palmer, MA (US)

(72) Inventors: Amparo CASANOVA, Palmer, MA (US); Stephen DONOGHUE, Palmer, MA (US); Alec MIAN, Palmer, MA (US); Gabriel BOUCHER, Palmer, MA (US)

(21) Appl. No.: 18/709,140

(22) PCT Filed: Nov. 14, 2022
(86) PCT No.: PCT/US2022/079813

§ 371 Date: May 10, 2024

Related U.S. Application Data
(60) Provisional application No. 63/363,774, filed on Apr. 28, 2022.
(60) Provisional application No. 63/264,092, filed on Nov. 15, 2021.
(60) Provisional application No. 63/263,969, filed on Nov. 12, 2021.

(51) Int. Cl. G16H 20/10; G16H 40/20

(52) U.S. Cl. CPC G16H 20/10; G16H 40/20

(57) ABSTRACT

A method of alerting to medication overuse headache risk is provided that includes providing a plurality of input prompts via a graphical user interface of a client device to receive medication usage data at a server system associated with at least one of the client device and a clinician device. The method also includes determining a trend of medication usage data indicating medication overuse known to cause a disease symptom onset. Based on the trend, an alert of medication overuse risk can be displayed via the graphical user interface on the client device and/or a clinician dashboard on the clinician device. Displaying the alert via the graphical user interface can include displaying a warning related to medication overuse and an instruction to seek medical advice. Displaying the alert via the clinician dashboard can include displaying the warning of medication overuse and medication type being overused.

### CROSS-REFERENCE TO RELATED APPLICATIONS

This application incorporates by reference in their entirety the disclosures of U.S. provisional application 63/263,969 filed on Nov. 12, 2021; and U.S. provisional application 63/264,092 filed on Nov. 15, 2021. This application further incorporates by reference in its entirety the disclosure of U.S. provisional application 63/268,225 filed on Feb. 18, 2022.

### BACKGROUND

Conventional techniques for users to manage medication usage include keeping a written record of times when medication was administered as well as the medication dosage. Other conventional techniques can include a user keeping an electronic diary of medication types, medication administration times, and medication dosage, along with perhaps other management related data. Some platforms for managing medication involve providing alerts to users when the medication was taken in the wrong dose or at the wrong time. However, these methods have proved inadequate in preventing medication overuse headache for a number of reasons, including sub-optimal record keeping and lack of coordination with clinician caregivers.

There is a need in this art for improvements to recording medication usage data and reporting the medication usage data to patients and clinicians, as well as providing alerts of risk of medication overuse based on the medication usage data.

### SUMMARY

This disclosure relates generally to methods and devices for implementing such methods for receiving patient data, including medication use data, determining the existence of a medication overuse risk based on medication usage trends, and alerting either the patient, a clinician attending the patient, or both of the medication overuse risk.

A first embodiment describes a method. The method includes providing a plurality of input prompts via a graphical user interface on a client device. The method further includes receiving medication usage data via the plurality of input prompts. The medication usage data can be received at a server system associated with at least one of the client device and a clinician device. The method also includes determining a trend of medication usage data that indicates medication overuse known to cause onset of a disease symptom. The method further includes displaying an alert of medication overuse risk based on the trend indicating medication overuse. The alert of medication overuse risk can be displayed via at least one of the graphical user interface on the client device and a clinician dashboard on the clinician device. Displaying the alert of medication overuse risk via the graphical user interface can include displaying a warning indication related to medication overuse and an instruction to seek medical advice. Displaying the alert of medication overuse risk via the clinician dashboard can include displaying the warning related to medication overuse and medication type being overused.

Within examples, a second embodiment describes a method including providing a plurality of input prompts via a graphical user interface of a client device. The method includes receiving medication usage data via the plurality of input prompts. The medication usage data can be received at a server system associated with the client device. The method also includes determining a trend of medication usage data that indicates medication overuse known to cause a disease symptom onset. The method further includes displaying an alert of medication overuse risk based on the trend indicating medication overuse. The alert of medication overuse risk can be displayed via the graphical user interface on the client device. Displaying the alert of medication overuse risk via the graphical user interface can include displaying a warning indication related to medication overuse and an instruction to seek medical advice.

Within examples, a third embodiment describes a method including receiving medication usage data at a server system associated with a clinician device. The method can also include determining a trend of medication usage data that indicates medication overuse known to cause a disease symptom onset. The method can further include displaying an alert of medication overuse risk based on the trend indicating medication overuse. The alert of medication overuse risk can be displayed on a clinician dashboard on the clinician device. Displaying the alert of medication overuse risk via the clinician dashboard can include displaying a warning indication related to medication overuse and medication type being overused.

The foregoing summary is illustrative only and is not intended to be in any way limiting. In addition to the illustrative aspects, embodiments, and features described above, further aspects, embodiments, and features will become apparent by reference to the figures and the following detailed description.

### BRIEF DESCRIPTION OF THE DRAWINGS

FIG. 1 illustrates a system for providing alerts of medication overuse risk, according to an example embodiment.

FIG. 2 illustrates an example client device and/or clinician device, according to an example embodiment.

FIG. 3 illustrates an example method, according to an example embodiment.

FIGS. 4A-4J illustrate a plurality of example input prompts via a graphical user interface, according to an example embodiment.

FIGS. 5A-5D illustrate an alert via a graphical user interface, according to an example embodiment.

FIGS. 6A-6E illustrate an alert via a graphical user interface, according to an example embodiment.

FIGS. 7A-7F illustrate an alert via a graphical user interface, according to an example embodiment.

FIGS. 8A-8D illustrate an alert via a graphical user interface, according to an example embodiment.

FIGS. 9A-9D illustrate an alert via a graphical user interface, according to an example embodiment.

FIGS. 10A-10B illustrate an alert via a clinician dashboard, according to an example embodiment.

FIGS. 10A-10B illustrate an alert via a clinician dashboard, according to an example embodiment.

FIGS. 11A-11B illustrate an alert via a clinician dashboard, according to an example embodiment.

FIGS. 12A-12B illustrate an alert via a clinician dashboard, according to an example embodiment.

FIGS. 13A-13B illustrate an alert via a clinician dashboard, according to an example embodiment.

FIGS. 14A-14B illustrate an alert via a clinician dashboard, according to an example embodiment.

FIG. 15 illustrates an example method, according to an example embodiment.

FIG. 16 illustrates an example method, according to an example embodiment.

FIG. 17 illustrates a clinician dashboard, according to an example embodiment.

FIGS. 18A and 18B illustrate a clinician dashboard, according to an example embodiment.

FIG. 19 illustrates a clinician status summary update, according to an example embodiment.

### DETAILED DESCRIPTION

Example methods and systems are described herein. It will be understood that the words “example,” “exemplary,” and “illustrative” are used herein to mean “serving as an example, instance, or illustration.” Any embodiment or feature described herein as being an “example,” being “exemplary.” or being “illustrative” is not necessarily to be construed as preferred or advantageous over other embodiments or features. The example embodiments described herein are not meant to be limiting. It will be readily understood that the aspects of the present disclosure, as generally described herein, can be arranged, substituted, combined, separated, and designed in a wide variety of different configurations, all of which are explicitly contemplated herein.

Currently, patients can use physical and virtual diaries to record medication usage. The recorded medication usage data is dependent upon the patient entering all medication use. However, there are times when a patient may not enter data. For example, patients could forget to enter data in a timely manner. Even after recording medication usage data, currently a patient may have to wait until the patient has an appointment with the clinician and the clinician has an opportunity to review the data to gain any understanding of how the medication usage data may be affecting the patient's life. Clinician appointments can be weeks if not months apart, leaving the patient without any answers or guidance. In addition, the clinician's advice on the medication usage data is reliant upon the accuracy of the data entered by the patient.

Furthermore, identifying risks, such as medication overuse, can take a long time to diagnose because of the magnitude of data that should be analyzed. One particular medication overuse scenario is medication overuse headache, described by the International Classification of Headache Disorders (ICHD) as headache occurring on 15 or more days per month in a patient with a pre-existing headache and that has developed as a consequence of regular overuse of acute or symptomatic headache medication for more than three months. Overuse of acute or symptomatic headache medication is defined (or “understood”) as using a medication on 10 or 15 or more days per month, (depending on the medication) for at least three months. For example, opioid overuse occurs from taking opioids 10 or more days per month. Medication overuse headache risk can occur when a patient has taken an opioid medication 10 or more days per month for three months. This pattern of medication use can cause migraine headaches. Currently, medication overuse headache is diagnosed only once it has already occurred by reviewing data gathered over the three-month period. Currently, there is no way to determine that a patient is at risk of medication overuse before medication overuse has occurred.

Provided herein are embodiments of the disclosed invention to identify and alert a trend indicating medication overuse and medication overuse headache risk. In the practice of the inventive methods, a plurality of input prompts can be provided to a patient by way of a graphical user interface of a client device. In example embodiments provided herein the client device can belong to a patient. By way of the input prompts, a server system can receive medication usage data. The server system can be associated with the client device and/or a clinician device. An example embodiment can include determining a trend in the medication usage data that indicates medication overuse known to cause a disease symptom onset; prior to the onset of the disease symptom. Based on the trend indicating medication overuse, an example embodiment of the method can include displaying an alert of medication overuse risk. The alert can be displayed via the graphical user interface on the client device and/or on a clinician dashboard on the clinician device. Displaying the alert of medication overuse risk via the graphical user interface on the client device can include displaying a warning indication related to medication overuse and an instruction to seek medical advice. Displaying the alert of medication overuse risk via the clinician dashboard on the clinician device can include displaying the warning related to medication overuse and medication type being overused.

As previously mentioned, typically it may take 90 days (i.e., 3 months) to diagnose medication overuse. However, using embodiments provided herein, an alert of medication overuse can be given in as little as 50 days. Additionally, an alert of medication overuse risk within the following two months can be given in as little as 10 days, and an alert of medication overuse risk within the following month can be given in as little as 40 days.

As used herein, a disease symptom is an observable manifestation of a particular disease or disorder. A disease symptom is observable by the person experiencing the symptom. In operation, a disease symptom can be characterized by multiple metrics, including but not limited to one or more of: (i) a time (or range of times) when the user experienced the disease symptom; (ii) a severity of the disease symptom; (iii) aspects or characteristics describing the disease symptom; and/or (iv) whether the disease symptom was accompanied by other related disease symptoms (and perhaps disease factors and/or disease triggers, which are described in further detail below). In non-limiting examples, where the disease is migraine and the disease symptom is a migraine headache, the characterization metrics for the migraine headache symptom can include any one or more of: (i) when the headache occurred; (ii) how long the headache lasted; (iii) the intensity and/or severity of the headache; (iv) the location of the headache along the user's head; and/or (v) whether the headache was accompanied by other related symptoms such as nausea or dizziness, and if so, the time, duration, intensity/severity of the accompanying symptoms. Disease symptoms for other chronic diseases can include different characterization metrics. More generally, a symptom can be a physiological or psychological manifestation that is not necessarily associated with a particular disease or disorder. For example, a user of a healthcare management application can experience a symptom without knowing, which, if any, known disease or disorder is causing the symptom.

As used herein, a disease factor is any event, exposure, action, or conduct related to and/or performed or experienced by a user that has the potential to influence, affect, or cause the user to experience a disease symptom, or in some cases, prevent the user from experiencing a disease symptom. Disease factors can include both: (i) voluntary or modifiable conduct and/or experiences by the user over which the user has at least some control, such as reacting to emotional states (anger, boredom, stress, anxiety, etc.), consumption of a particular food product, ingestion of a particular therapeutic agent, application of a particular therapeutic agent, ingestion of a particular dietary supplement or drug, performance of a particular physical activity, and/or exposure to a particular chemical agent; and (ii) involuntary or un-modifiable conduct and/or experiences, such as unavoidable exposure to environmental factors (e.g., smog, sunlight, rain, snow, high or low humidity, or high or low temperatures), ingestion or other exposure to mandatory therapeutic agents or drugs (e.g., drugs to treat or maintain other diseases), and effects of other diseases or physical conditions over which the user has little or perhaps effectively no control.

Like a disease symptom, a disease factor can also be characterized by multiple metrics, and different disease factors can have different characterization metrics. For example, for a food- or drug consumption-based disease factor, the characterization metrics can include, for example: (i) when the user consumed the food or drug; and/or (ii) how much of the food or drug the user consumed. Characterization metrics for an exposure-based disease factor can include, for example: (i) when the user was exposed; (ii) the intensity (e.g., bright sunlight) of the exposure; and/or (iii) the duration of the exposure.

In some embodiments, disease factors can also include premonitory symptoms or warning signs that do not actually cause the user to experience a disease symptom but are closely associated with onset of a disease symptom for a particular user. Thus, disease symptoms may be associated with one or more premonitory symptoms. For example, in migraine a premonitory symptom can be a craving for sweet foods before the user experiences the migraine headache. In this example, the sweet craving does not cause the migraine, but instead is likely related to some physiological change associated with the migraine attack.

In some instances, a particular physical manifestation felt by the user can be a disease symptom or a disease factor depending on its position in the physiological pathway. To use obesity as an example, reduced physical activity can be a disease factor because it tends to cause a disease symptom associated with obesity (e.g., excess fat stores). But in a different pathway, reduced physical activity can be a disease symptom that is caused by, for example, osteoarthritis that often follows obesity due to increased fat mass.

In some contexts, symptoms can be observed in a user that are not associated with a particular disease. For example, a user can wish to track a particular symptom that she has identified as her “most bothersome symptom” (i.e., a symptom that the user feels is most disruptive or distracting out of a set of symptoms that the user might experience). In these examples, a user can track the symptom itself, along with potential factors that can be associated with the symptom. In this manner, statistical associations can be formed between various symptoms and factors without each symptom necessarily corresponding to a particular disease for an individual. Accordingly, as used herein, the term “symptom factor” can be any factor which relates to a symptom of a specific disease, but which precedes the ‘main’ or ‘most bothersome’ symptom. For example, “symptom factor” can be related to or include any event, exposure, action, or conduct related to and/or performed or experienced by a user that can influence, affect, or cause the user to experience a symptom, or in some cases, prevent the user from experiencing a symptom, without the symptom necessarily being associated with a particular disease or disorder. It should be understood that the disease factors described herein and defined above can more generally be referred to as symptom factors. Accordingly, any forthcoming description that involves disease factors as they pertain to disease symptoms can more generally be understood as symptom factors and corresponding symptoms. In other words, symptom factors may be the factors that contribute to causing a corresponding symptom of a disease.

A disease trigger is a disease factor that has been determined, for example through statistical analyses or other methods, to have a sufficiently strong association with a particular disease symptom for an individual user so as to become user-specific information of high interest and clinical use to the user and their clinician. In some cases, a disease trigger can be strongly associated with causing the user to experience the particular disease symptom, or at least increasing the risk or likelihood that the user will experience the particular disease symptom. In other cases, its opposite, a disease protector, can be strongly associated with preventing the user from experiencing the particular disease symptom, or at least decreasing the risk or likelihood that the user will experience the particular disease symptom; such disease triggers can be referred to herein as protectors because they tend to reduce the user's likelihood of experiencing the disease symptom. In some embodiments, a disease trigger for a user is a disease factor having a determined univariate association with a disease symptom for the user, where the determined univariate association demonstrates a statistically significant hazard ratio or odds ratio (greater than 1) or equivalent regression coefficient (greater than 0) at a fixed significance level (e.g., p-value less than 0.05) in univariate regression models or in equivalent multivariable models.

In some embodiments, one or more server systems analyze disease symptom and disease factor data received from a patient population to determine which disease factors rise to the level of disease triggers for a particular patient. In operation, a patient population may include many (hundreds, thousands, or perhaps millions) of patients who all share one or more similarities (e.g., the same age or age range, same gender, same ethnicity, same national origin, suffer from the same disease, have the same allergies, have the same genetic markers, and/or perhaps other similarities). Some patients may be members of multiple patient populations.

Some embodiments generally apply a two-step iterative approach to identify disease factors and triggers for a patient population, and then (based on the identified disease factors and triggers for the patient population) identify disease factors and triggers for an individual patient. For the first step, the server systems collect and analyze disease factor data and disease trigger data from patients in a patient population to identify the disease factors that tend to be most strongly associated with a particular disease symptom for the patients in the patient population Client devices (under direct or indirect control of the server systems) are configured to prompt patients in the patient population to enter characterization metrics for the disease factors that the server systems have determined to be most strongly associated with the particular disease symptom for the patient population. For the second step, the server systems analyze the disease factor characterization metrics for the patients in the patient population, and for each patient in the population, the server systems determine the strength of the association (for that patient) between particular disease factors and the disease symptom. Then, for each patient, the server systems designate the disease factors that are most strongly associated with the disease symptom as disease triggers for the patient. The process is iterative in that disease triggers identified for one patient in a patient population can be analyzed for the whole patient population and then tested for individual patients. Some aspects of this iterative, two-phase process are described in PCT Application PCT/US2014/013894 filed on Jan. 30, 2014, the contents of which are incorporated herein by reference.

For most users, many commonly suspected disease factors have turned out to not be disease triggers, i.e., the most suspected disease factors are neither triggers nor protectors for the user. However, there is a high degree of heterogeneity between users with respect to which disease factors affect different users as either disease triggers or disease protectors. To help a user manage his or her disease, the user advantageously wants individualized information about which suspected disease factors are disease triggers/protectors for them and which suspected disease factors have no effect for them. A suspected disease factor that has no effect for that individual is referred to herein as a “no-association factor” or NAF.

As used herein, a phase of a particular symptom can refer to a discrete period in which a particular physical manifestation or manifestations are expected in relation to a given symptom. Some symptoms may manifest in a cyclical manner denoted by a plurality of recurring phases. For example, migraine headaches, Irritable Bowel Syndrome (IBS), and asthma each have episodic recurrences characterized by two or more phases of experiencing the symptom. In the context of the present disclosure, experiencing a disease symptom can be understood as experiencing a level of severity of the disease symptom, and can include a level of severity relative to prior periods of experiencing the symptom. For example, this phenomenon can manifest as experiencing a worsening or improvement in the disease symptom relative to another phase or a past cycle. Other symptoms denoted by such episodic recurring phases are contemplated in the context of the following disclosure. As an example, related to recurring phases, an interictal phase between migraine attacks can typically be associated with no manifestations of a migraine headache or corresponding disease symptoms. However, such phases might not be tied to biomarker levels in any discrete way. For example, it has been shown that the consumption of chocolate has no effect on migraine attacks. For many symptoms, different phases can be sequential and cyclical, and can be predictable in terms of order (e.g., phase 1 always precedes phase 2, etc.) even if they are less predictable in terms of duration (e.g., how long phase 3 will last for a particular cycle).

In some embodiments described herein, a biomarker can be used to predict the occurrence of symptoms. A biomarker can refer to a measurable substance in a user, or physiological state of the user, or a perceived stressor experienced by the user, or a specific combination of these, whose presence (or a degree thereof) is indicative of one or more phenomena experienced by the user. For example, a “substance” in this context can be a detectable molecule produced by the human body that can be measured using a sensor device. A “physiological state” can include a condition or state of the body or bodily functions of a user. A “stressor” can be an emotional or physical prompt experienced by a user or a physiological response to such a prompt. For example, a “stressor” in the context of a migraine can include one or more of stress, anxiety, heart rate variability, breathing pattern, hearing sensitivity, and sleep quality. Other examples of biomarkers are possible as well. A biomarker can be more broadly understood as including biological signs of a given disease or condition. Within examples described herein, a biomarker level, and observed patterns in such biomarker levels during phases of a symptom, can be leveraged to predict the symptom and to recommend an intervention to prevent or mitigate the predicted symptom Different biomarkers or combinations of biomarkers can be predictive of a symptom for different users.

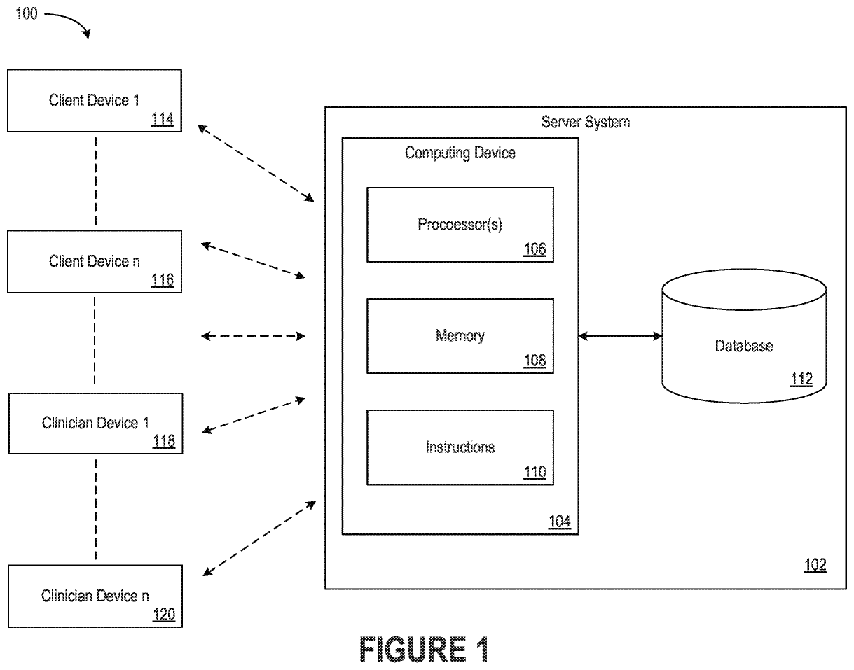

FIG. 1 shows a system 100 for providing alerts related to medication overuse and predictions and recommendations related to the onset of one or more symptoms to a user population. System 100 includes a server system 102 of the health management platform including a computing device 104, a plurality of client devices that are communicatively coupled to the computing device 104, a plurality of clinician devices that are communicatively coupled to the computing device 104, and a database 112. The plurality of client devices includes client device 114 and client device 116. The plurality of clinician devices includes clinician device 118 and clinician device 120.

Computing device 104 includes one or more processor(s) 106, a memory 108, and instructions 110. The processor(s) 106 can include, for example, central processing unit(s) or CPU(s), one or more general purpose processors, or one or more specialized processors. Memory 108 can include a tangible non-transitory computer readable memory. The instructions 110 can be stored on the memory 108 and be executable by the processor(s) 106 to perform functions associated with computing device 104. Though computing device 104 is depicted as a single device, it should be understood that computing device 104 can represent one or more servers, a cloud-based processing system, or a plurality of computing devices operating collectively within or in association with server system 102 of the health management platform. Further, while database 112 is depicted as being separate from computing device 104, it should be understood that, in some contexts, database 112 can be incorporated into computing device 104, perhaps as part of the memory 108.

The plurality of client devices can include one or more client devices associated with users of a healthcare management application who are patients (e.g., a client device might be a smartphone or tablet controlled by a user). The plurality of client devices may include or be communicatively coupled to one or more sensor devices configured for determining biomarker levels for a given user (e.g., a heartrate monitor, electrocardiogram sensor, blood pressure sensor, etc.). The client device can include one or more processor(s), a memory, and instructions.

The plurality of clinician devices can include one or more clinician devices associated with users of a healthcare management application who are healthcare providers (e.g., a clinician device might be a smartphone or tablet controlled by a user). The clinician device can include one or more processor(s), a memory, and instructions.

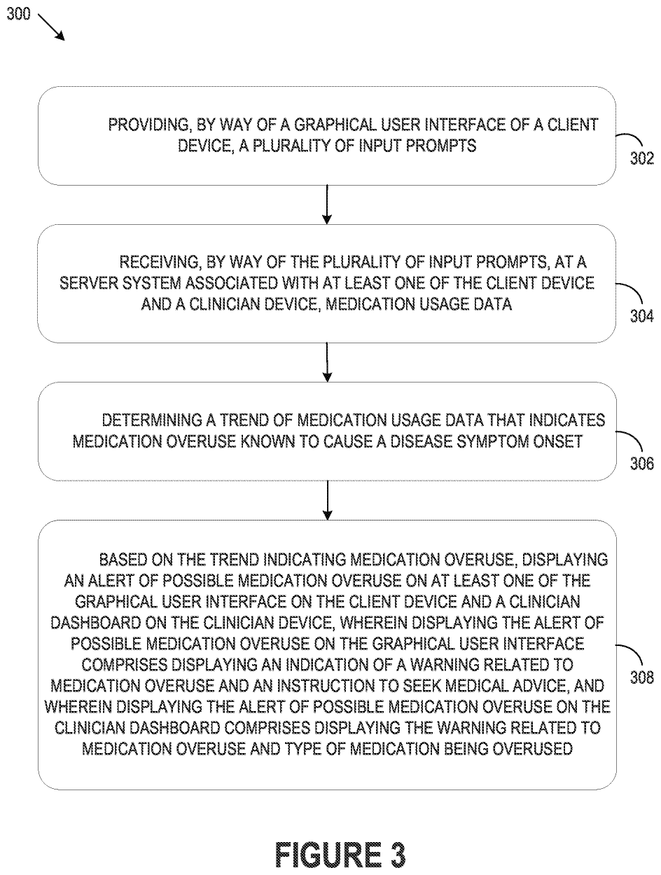

In some examples, computing device 104 can be associated with hosting and/or configuring a health management application deployed on one or more client devices Computing device 104 can receive inputs (e.g., inputs entered into the healthcare management application via a client device or data from a sensor device) that forms a dataset for use in a statistical analysis of one or more symptoms of a user population. The user population can include a plurality of users associated with different accounts in the health management application. Computing device 104 can be configured to perform this statistical analysis using a machine learning model, and can have an architecture designed for such modeling. For example, computing device 104 can be implemented with a deep learning architecture that uses a neural network to analyze the inputs and provide an output based on prior training. For example, the neural network can be trained to predict the onset of one or more symptoms for users in the user population in a supervised, semi-supervised, or unsupervised manner. Other machine learning techniques can be implemented in computing device 104. In alternative examples, computing device 104 can make predictions for the user population using regression analyses. Other ways of determining predictions for the user population are possible. Further, computing device 104 or a client device can correspondingly recommend one or more actions of a user based on a predicted symptom. Examples of these determinations and functions are described below with respect to FIGS. 3-6.

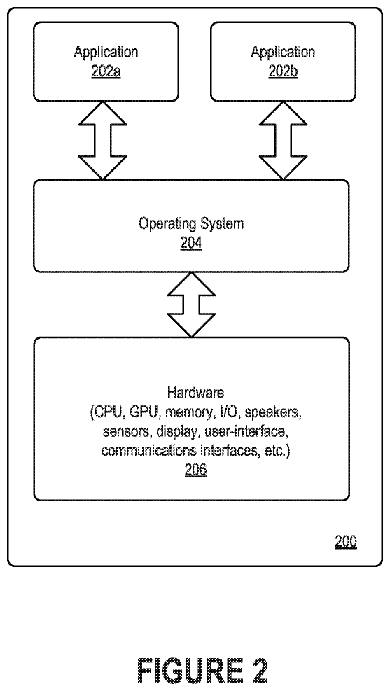

FIG. 2 shows an example device 200 according to an example embodiment. The device can be a client device and/or a clinician device. The device 200 can be a smartphone, tablet, desktop or laptop computer, or any other type of computing device with the capability of generating, gathering, and/or presenting data disclosed and described herein to a user as well as performing any ancillary functions that can be required for effective implementation of the user population segmentation, symptom prediction, and recommendation methods disclosed and described herein.

Device 200 includes hardware 206 comprising: (i) one or more processors (e.g., a central processing unit(s) or CPU(s) and/or graphics processing unit(s) or GPU(s)); (ii) tangible non-transitory computer readable memory (otherwise referred to as non-transitory computer readable media); (iii) input/output components (e.g., speaker(s), sensor(s), display(s), headphone jack(s) or other interfaces); and (iv) communications interfaces (wireless and/or wired). The hardware 206 components of the computing device 202 are configured to run software, including an operating system 204 (or similar) and one or more applications 202a, 202b (or similar) as is known in the computing arts. One or more of the applications 202a and 202b can correspond to computer-executable program code that, when executed by the one or more processors, cause the client device 200 to perform one or more of the functions and features described herein, including but not limited to any (or all) of the features and functions of methods 300, 1500, and 1600 disclosed below, as well as any other ancillary features and functions known to persons of ordinary skill in the computing arts that can be required or at least desired for effective implementation of the features and functions of methods 300 and 600, even if such ancillary features and/or functions are not expressly disclosed herein.

Method 300 is a flow chart of a method of displaying an alert of medication overuse risk via at least one of the graphical user interface on the client device and/or the clinician dashboard on the clinician device. Medication overuse risk can occur when the patient has overused a medication for at least three months. Method 300 may include one or more operations, functions, or actions, as depicted by one or more of blocks 302, 304, 306, and 308 each of which may be carried out by any of the systems shown in prior figures, among other possible systems.

Those skilled in the art will understand that the flow charts described herein illustrate functionality and operation of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art.

At block 302, method 300 involves providing via plurality of input prompts via a graphical user interface of a client device. The plurality of input prompts can be displayed via the graphical user interface. In an example embodiment, the graphical user interface can be part of an application on the client device. Each time the application is opened on the client device, the graphical user interface can automatically display a request to enter data in the form of a plurality of input prompts.

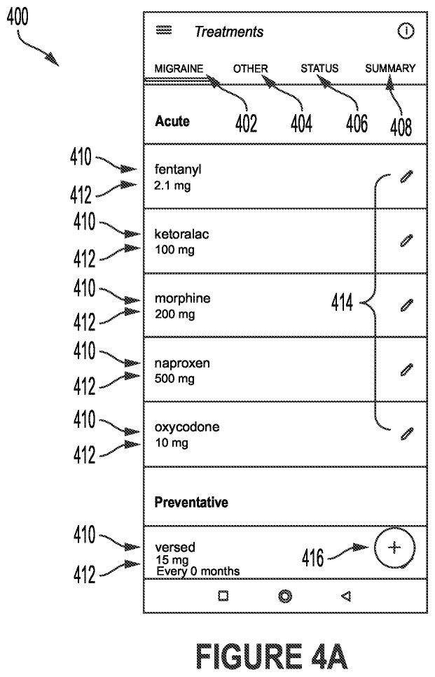

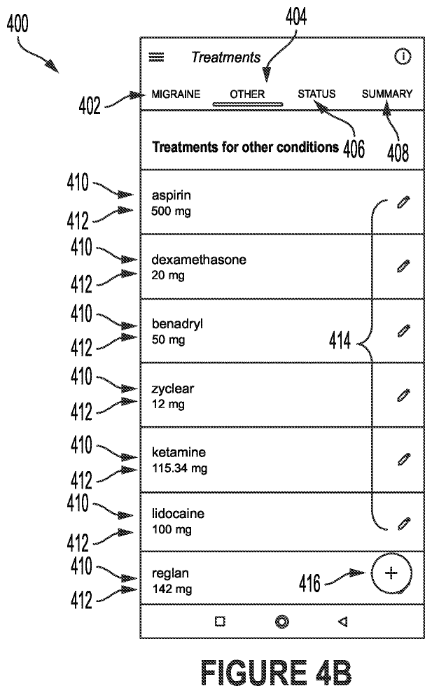

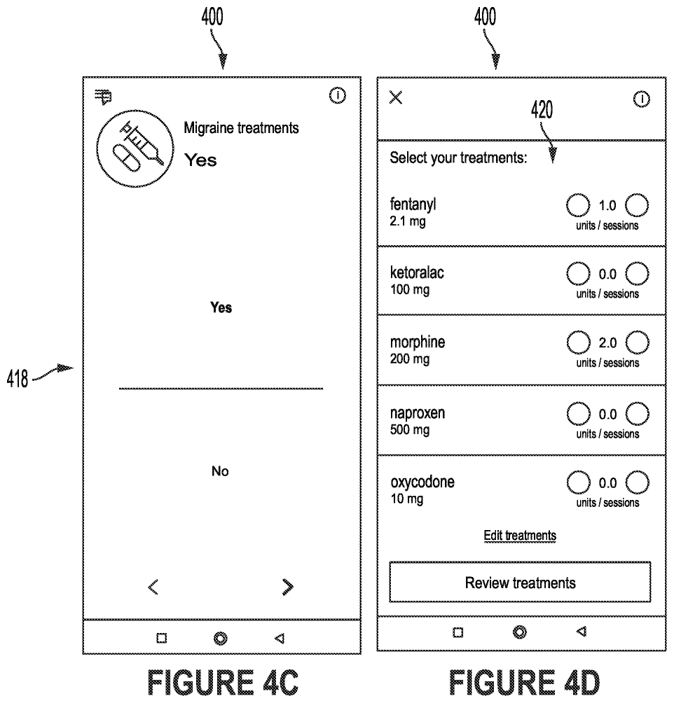

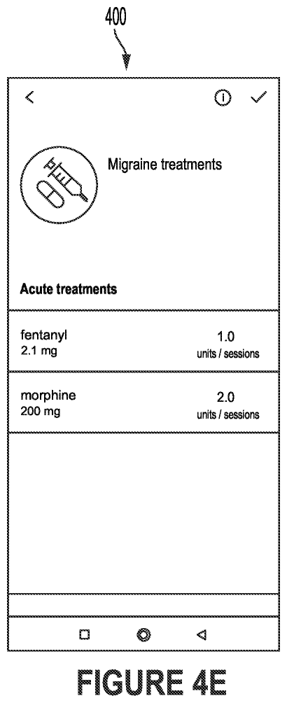

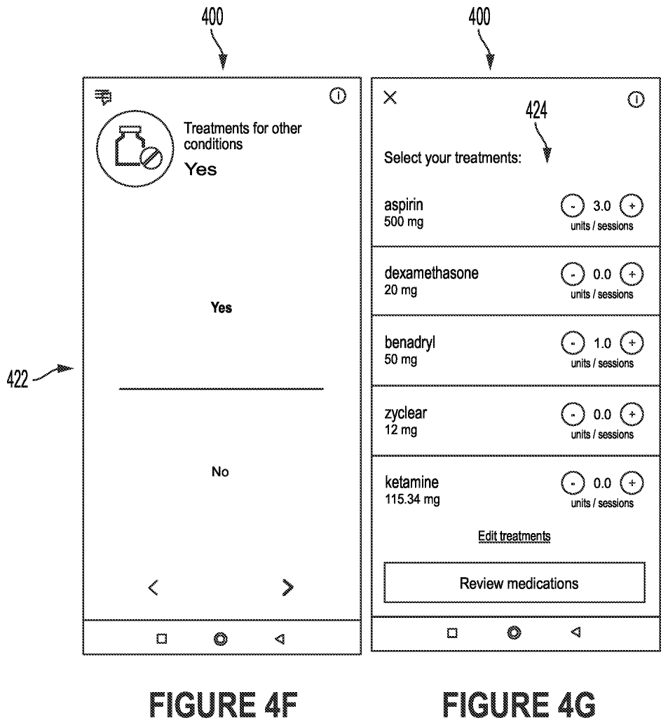

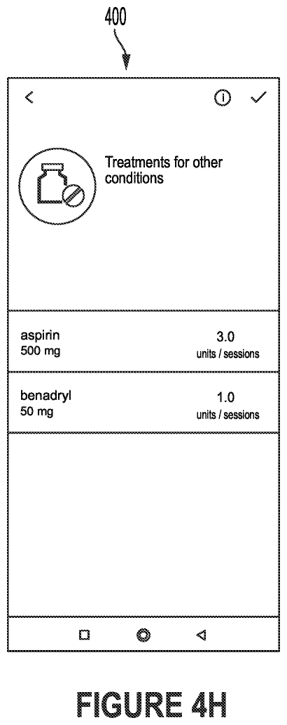

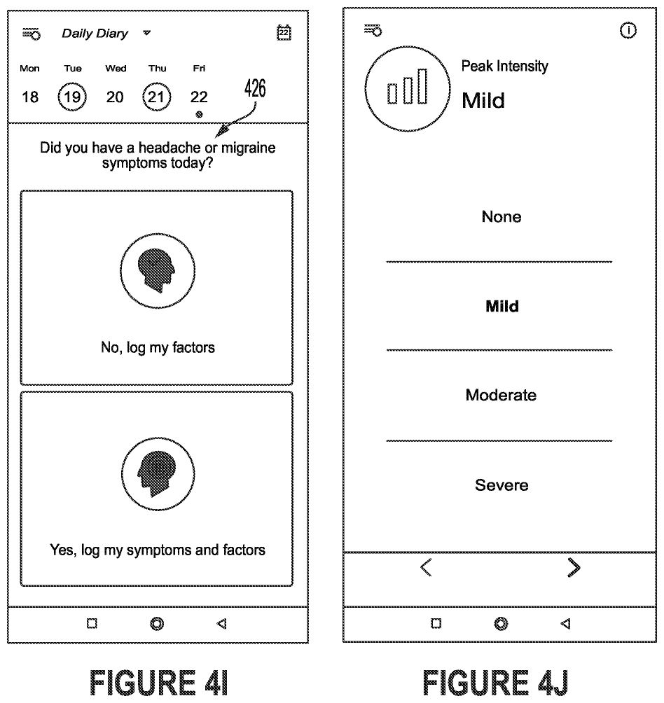

FIG. 4A and FIG. 4B illustrate the plurality of example input prompts for a patient to enter medication usage data via the graphical user interface 400 of the client device. As illustrated in the Figures, the patient can navigate to a treatment menu via the graphical user interface. In the treatment menu there can be submenus including but not limited to a migraine medication menu 402, an ‘other’ medication menu 404, a medication status menu 406, and a summary menu 408. The migraine medication menu and the other menu can include a plurality of medications 410 and the dosage 412 associated with each. The medication and their associated dosage and strength can be added by selecting icon 416. Furthermore, the medications and their dosages can be edited or removed by selecting an edit icon 414 via the graphical user interface.

In an example embodiment, the input prompts can include a request for data relating to days with intake of symptomatic headache medications, at least one medication type, medication dosage, medication administration route, and total number of medication doses taken in a day, days with headache, type of headache, and severity of headache. FIGS. 4C, 4D, and 4E illustrate how daily medication use for migraine treatment can be tracked using the plurality of input prompts via the graphical user interface 400 of the client device. As illustrated in FIG. 4C, an input prompt 418 can include displaying a prompt related to whether or not a migraine treatment was used. The input prompt displayed on the graphical user interface can read “Did you use any medication or other therapies to treat your headaches/migraine attacks?”. The patient can select yes or no in response to these prompts. If yes is selected, the patient may be instructed to enter the medication or other therapies used. FIG. 4D illustrates that when the patient selects, via the graphical user interface, that a migraine treatment was used, the graphical user interface can display a medication input prompt 420. The medication input prompt 420 can allow the user to select the types and amount of treatments that the patient used. The patient may be able to select medications previously entered into the application as in FIGS. 4A and 4B. As illustrated in FIG. 4E, the selections can be displayed via the graphical user interface. In an example embodiment, the application can display the plurality of input prompts on the client device at predetermined times. For example, the plurality of input prompts can be displayed early in the morning when a user is waking up, or late at night before the user goes to bed. In an additional embodiment, the graphical user interface can also include an icon for entering data. When the icon is selected, the graphical user interface can display the plurality of input prompts.

In an example embodiment, the input prompts can be further used for entering treatments for conditions that are not related to headache. FIGS. 4F, 4G, and 4H illustrate how daily medication use for other conditions can be tracked using the plurality of input prompts via the graphical user interface 400 of the client device. As illustrated in FIG. 4F, an input prompt 422 can include displaying a prompt related to whether or not a treatment was used. The input prompt displayed on the graphical user interface can read “Did you use any medications or treatments for something other than headache/migraine?”. The patient can select yes or no in response to these prompts. If yes is selected, the patient may be instructed to enter the medication or other therapies used. FIG. 4G illustrates that when the patient selects, via the graphical user interface, that a treatment was used, the graphical user interface can display a treatment input prompt 424. The treatment input prompt 424 can allow the user to select the types and amount of treatments that the patient used. The patient may be able to select medications previously entered into the application as in FIGS. 4A and 4B. As illustrated in FIG. 4H, the selections can be displayed via the graphical user interface.

In an example embodiment, the input prompts can also be used to enter days with headache, type of headache, and severity of headache. FIGS. 4I and 4J can illustrate how headaches and headache symptoms can be tracked using the plurality of input prompts via the graphical user interface 400 of the client device. As illustrated in FIG. 4I, an input prompt 426 displayed on the graphical user interface can ask whether the patient bad any headache or migraine symptoms on that day. The patient can select yes or no in response to these prompts. If yes is selected, the patient may be instructed to enter the intensity of the migraine or headache symptom. FIG. 4J illustrates that the patient can select at least one of “none”. “mild”, “moderate”, or “severe” to illustrate the intensity of the patient's headache or migraine.

At block 304, method 300 includes receiving medication usage data via the plurality of input prompts at a server system associated with at least one of the client device and/or a clinician device. As previously discussed, the plurality of input prompts can facilitate the gathering of medication usage data and send the medication usage data to the server system. The server system can be a centralized server system associated with one or both of the clinician device or the client device. The clinician device and/or the client device can transfer data back and forth via the server system. Alternatively, the clinician device or the client device could be associated with different servers. For example, the clinician device can be associated with the server system and the client device can be associated with a second server, or vice versa.

In an example embodiment, the medication usage data includes days with intake of symptomatic headache medications, at least one medication type, medication dosage, medication administration route, and/or total number of medication doses taken in a day. Symptomatic headache medication (including acute headache medication) can be found in the ICHD headache guidelines, and will also be known by a person having ordinary skill in the art. The medication type can include at least one of prescribed medication and/or non-prescribed medication.

In an example embodiment, the medication usage data can also be obtained from a medical device. For example, a device can be used to measure drug levels in a medication user's body. An insulin pump can monitor a patient's blood sugar and insulin levels and can send the blood sugar and insulin data to the server system. In an additional embodiment, a medical device can be on a medication bottle and can track when the bottle is opened to determine when medication was used and send it to the server system. In some embodiments, the patient may not have to enter data into the graphical user interface because the medical device can automatically detect physiologic inputs (e.g., when a patient opens the graphical user interface on a smart watch the pulse or other physiological inputs are detected and entered without user intervention)

At block 306, method 300 includes determining a trend of medication usage data that indicates medication overuse known to cause a disease symptom onset. A person of ordinary skill in the art would understand that factors contributing to medication overuse known to cause a disease symptom onset can be found in the International Headache Society Classification ICHD guidelines and that the standards can change as the guidelines are updated.

In an example embodiment, certain medications can be ignored when making the determination of the trend. A clinician can enter a request through the clinician dashboard on the clinician device to disregard the at least one medication type when determining the trend of medication usage data. An example embodiment can include receiving, at the server system, the request to disregard the at least one medication type when determining the trend of medication usage data. When the request is received, the method can include disregarding the at least one medication type while determining the trend of medication usage data. For example, low dose aspirin can be disregarded as it is not a factor in contributing to medication use headache. In an additional example, the disregarded medication could be a medication that contributes to medication overuse headache if the medication is required for another condition during a possibly limited period of time (e.g. after a surgery).

In an example embodiment, the trend can be determined based on data compiled over a period of time. The server system can receive the medication usage data over a survey period via the plurality of input prompts. The trend of medication usage data that indicates medication overuse known to cause a disease symptom onset is determined over the survey period. Patient intake of symptomatic headache medications over the survey period can be received at the server system associated with the client device. At the server system, it can be determined that daily usage of the medication is over a threshold of recommended medication usage days over the survey period. For example, for opioids, triptans, ergotamines, combination analgesics or multiple medications on the same day, the threshold can be taking medication on 10 days or more over the survey period. The threshold of 10 days may be used throughout, however a person of ordinary skill in the art would understand that the threshold can change. Particularly, the threshold can change depending on the medication type. For example, for non-opioid analgesics alone (ibuprofen alone, acetaminophen alone) the threshold can be 15 days. Further, the types of medications used in the method and the thresholds for each can be modified depending on the medications and related thresholds set by the International Headache Society in the ICHD.

In an example embodiment, the threshold can be programmable. The thresholds can be changed depending on the medication type and/or depending on the patient A patient who has developed chronic medication overuse headaches could need a lower threshold of recommended medication usage days over the survey period than the threshold recommended in the ICHD guidelines in order to protect them from medication overuse headache.

In an example embodiment, the survey period can be one month (e.g., 30 consecutive calendar days). The number of days in a month can be any number of days that a person of ordinary skill in the art would associate with a month. For example, a month can be anywhere from 28 days to 31 days. In an alternative embodiment, the survey period can be a programmable number of days set by the clinician or the International Headache Society.

In an example embodiment, the one-month survey period can be a sliding window that captures the worst-case scenario for medication overuse. The standard for alerting a patient to a risk of medication overuse headache due to opioids is that the patient has taken the opioid on at least 10 days in a first month, 10 days in a second month, and 10 days in a third month. However, the sliding window can be implemented to possibly shorten the amount of time needed in order to alert a patient to risk of medication overuse headache. For example, in the first month survey period once 10 days of medication use has been tracked the sliding window can be used so that days from before the tracking started are included in the survey period to reach the number of days needed for one month. The sliding window can be used to construe a worst-case scenario to warn the patient about medication overuse risk.

In an example embodiment, the warning related to medication overuse includes a warning that the trend of medication overuse may indicate a risk of disease symptom onset within two months. The disease symptom onset can include the patient developing a new headache when the patient has already been experiencing a pre-existing primary headache. A “new” headache can be a headache that has different characteristics from pre-existing primary headaches the patient has previously experienced. Different headache characteristics can be type of pain, level of pain, and location of pain. The standards for determining that a new headache may be caused by medication overuse includes that the patient overused medication for at least three months prior to the new headache's onset. Therefore, after the first survey period of one month, the patient can be warned that the trend indicates that in two more months the patient is at risk of developing disease symptoms associated with medication overuse headache, assuming that the next two months include medication overuse as well.

In an example embodiment, the survey period can include two months (e.g., a first month and a second month). The number of days in two months can be any number of days that a person of ordinary skill in the art would associate with two months. For example, two months can be anywhere from 56 days to 62 days. The number of days in each month can be any number of days that a person of ordinary skill in the art would associate with a month. For example, a month can be anywhere from 28 days to 31 days.

In an example embodiment, the two-month survey period can include the month-long sliding window previously discussed and a second month-long window. As previously stated, the standard for alerting a patient to medication overuse headache risk due to opioids is that the patient has taken the opioid on at least 10 days in a first month. 10 days in a second month, and 10 days in a third month. In the first month of the survey period, once 10 days of medication use has been tracked the sliding window can be used so that days from before the tracking started are included in the survey period to reach the number of days needed for one month. The first day of the second month of the survey period can be on the day following the tenth day of medication use in the first month of the survey period. The second month can be 28 days to 31 days long depending on the number of days selected for the first month.

During or at the end of the two-month survey period in which the patient overused medication in both the first month and the second month, the warning related to medication overuse can include a warning that the trend of medication overuse may indicate a risk of disease symptom onset within one month. The warning can indicate that if the patient overuses medication for one more month, the patient can develop disease symptoms including a new headache. The warning can appear after the patient has used medication on 10 or more days in the second month.

In an example embodiment, the survey period can include three months (e.g., a first month, a second month, and a third month). The number of days in three months can be any number of days that a person of ordinary skill in the art would associate with three months. For example, three months can be anywhere from 84 days to 93 days. The number of days in each month can be any number of days that a person of ordinary skill in the art would associate with a month. For example, a month can be anywhere from 28 days to 31 days.

In an example embodiment, the three-month survey period can include the month-long sliding window and the second month-long window previously discussed. The three-month survey period can also include an additional month-long sliding window. As previously stated, the standard for alerting a patient to medication overuse risk headache due to opioids is that the patient has taken the opioid on at least 10 days in a first month, 10 days in a second consecutive month, and 10 days in a third consecutive month. In the first month of the survey period, once 10 days of medication use has been tracked the sliding window can be used so that days from before the tracking started are included in the survey period to reach the number of days needed for one month. The first day of the second month of the survey period can be on the day following the tenth day of medication use in the first month of the survey period. The second month can be 28 to 31 days long depending on the number of days selected for the first month. The first day of the third month of the survey period can be on the day following the end of the second month. In the third month survey period once 10 days of medication use have been tracked the sliding window can be used so that it includes future days in the survey period in order to reach the number of days needed for one month.

During or after the three-month survey period in which the patient overused medication in the first month, the second month, and the third month the warning related to medication overuse can include a warning that the trend of medication overuse may indicate a risk of disease symptom onset. The warning can indicate that due to medication overuse for at least three months, the patient may experience disease symptoms including a new headache.

In instances where the patient has not overused medication in the month, either no alert will be displayed for the patient, or the alert from the previous month will be displayed. For example, if the patient has overused medication in the first month, but not in the second month, then the display may still indicate a risk of disease symptom onset was detected in the first month.

At times, a patient may not enter medication usage data. In those cases, the method may not be able to accurately provide a warning that the trend indicates a risk of medication overuse. In an example embodiment, the method can include determining at least one missing day of medication usage from the daily usage over the survey period. This can be determined as the days in which a patient did not enter any data. In one embodiment, the method can assume the worst-case scenario that on the days where data is missing the patient used medication. The assumed worst case scenario medication data will be factored into determining the trend. In an alternative example embodiment, the method can include predicting, using a trained machine learning model, daily usage for the at least one missing day of medication usage. The trained machine learning model can be trained on the medication usage data previously entered by the patient. The method can further include determining the trend of medication usage data that indicates medication overuse known to cause a disease symptom onset based on the predicted daily usage.

At block 308, method 300 includes based on the trend indicating medication overuse, displaying an alert of medication overuse risk via at least one of the graphical user interface on the client device and/or a clinician dashboard on the clinician device. The method 300 can include that displaying the alert of medication overuse risk via the graphical user interface includes displaying a warning indication related to medication overuse and an instruction to seek medical advice. The method 300 can also include displaying the alert of medication overuse risk via the clinician dashboard including displaying the warning related to medication overuse and medication type being overused.

In an example embodiment, when the trend is determined that indicates something related to medication overuse, an alert of medication overuse risk can be displayed on the client device belonging to a patient via the graphical user interface of and/or on the clinician device belonging to the clinician via the clinician dashboard. The patient can interact with data, enter data, and interact with the alerts via the graphical user interface. Similarly, the clinician can interact with and review the medication usage and other data via the clinician dashboard.

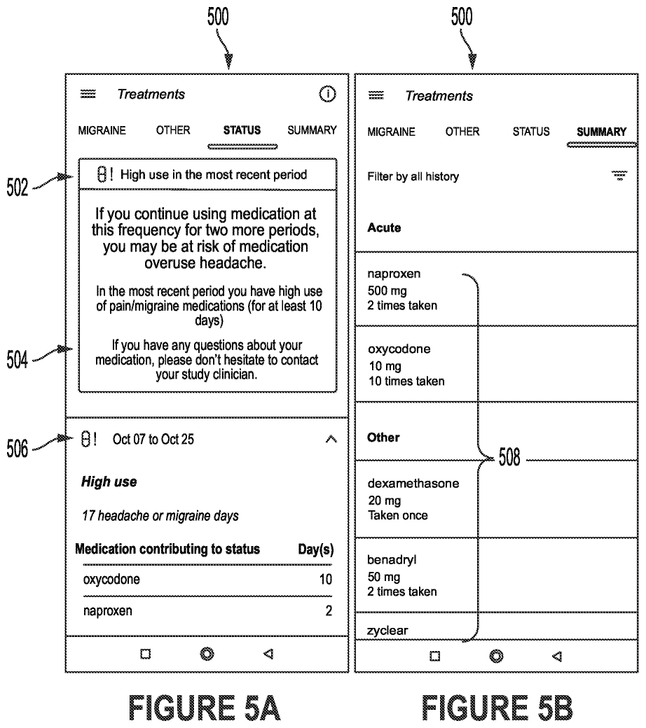

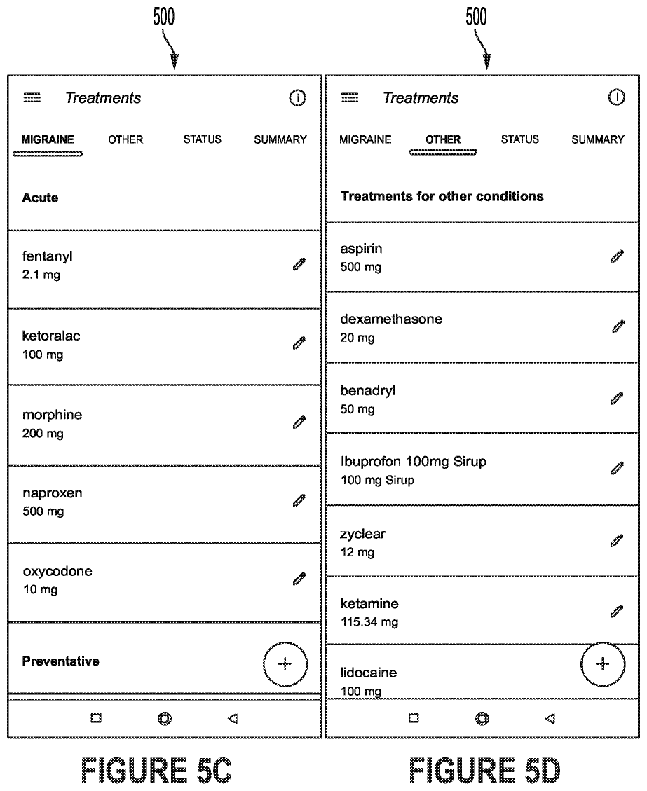

FIG. 5A illustrates an example embodiment of displaying the alert of medication overuse risk 502 via the graphical user interface 500 when the patient has overused medications in the most recent survey period (e.g., for one month) As illustrated, the alert 502 on the graphical user interface 500 can include displaying a warning indication related to medication overuse and an instruction to seek medical advice. Particularly, as illustrated in FIG. 5A, the warning can indicate that high medication use has been determined for the most recent survey period, and that if the patient continues to use the medication at the current frequency (e.g., following this trend) for two more periods, the patient may be at risk of medication overuse headache. The display can also include an instruction to seek medical advice 504. Additionally, the display can include an explanation of why there is an alert by explaining that in the most recent period the patient has had high use of pain/migraine medications (for at least 10 days or 15 days depending on the medication as previously discussed).

In an additional embodiment, the display via the graphical user interface 500 of the client device can include the dates 506 of the survey period and the type and amount of each medication type used during the survey period. Additionally, the display can include the number of days on which the patient experienced a headache or a migraine.

In an example embodiment, as illustrated in FIG. 5B, the display of the graphical user interface can include a summary with medication information 508. The medication information can include the different types of medication that the patient used during the survey period, the dosage of each medication, and the number of days the patient used the medication. As illustrated in FIGS. 5C and 5D, the display can also include a listing of different possible medications and the dosage of each medication similar to the display 400 in FIGS. 4A and 4B. The different medications can be listed under the “migraine” and “other” sub-menus and can include the dosages for each medication.

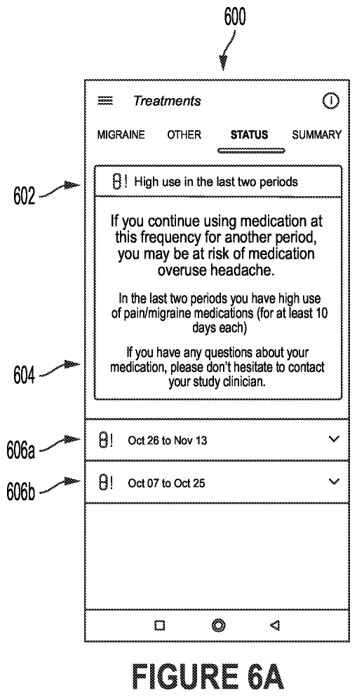

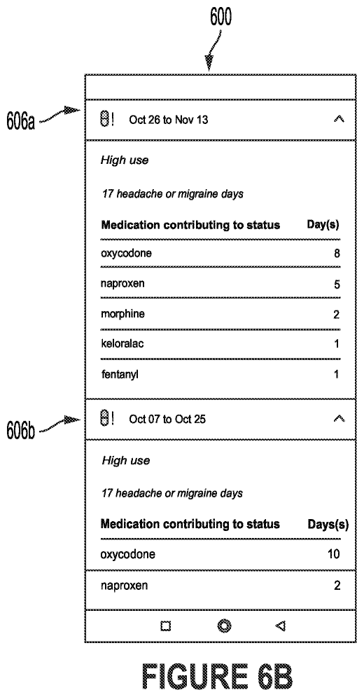

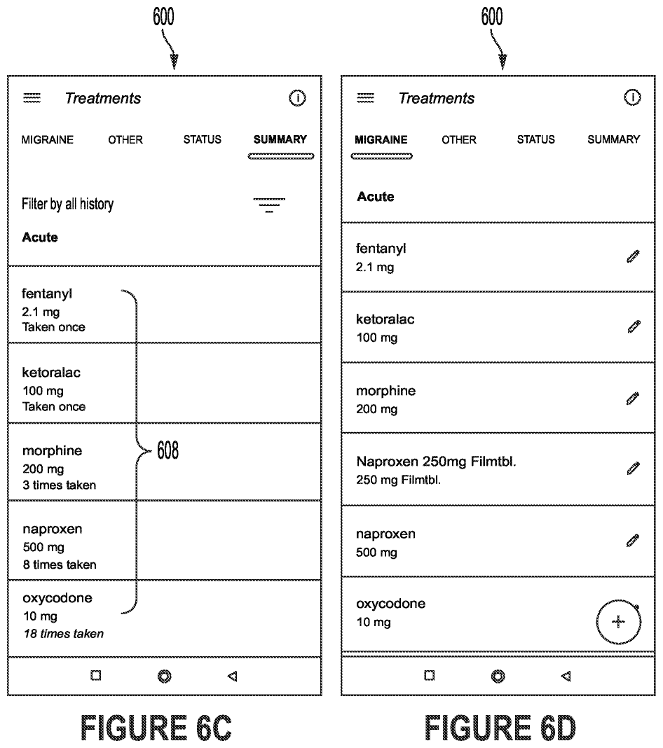

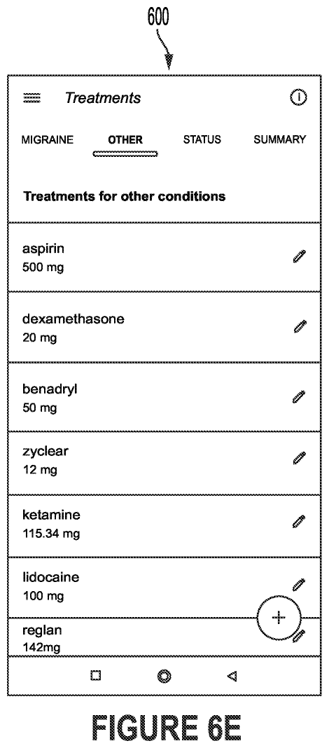

FIG. 6A illustrates an example embodiment of displaying the alert of medication overuse risk 602 via the graphical user interface 600 when the patient has overused medications for the most recent two survey periods in a row (i.e., for two months) As illustrated, the alert 602 on the graphical user interface 600 can include displaying a warning indication related to medication overuse and an instruction to seek medical advice. Particularly, as illustrated in FIG. 6A, the warning can indicate that high medication use has been determined for the two most recent survey periods, and that if the patient continues to use the medication at the current frequency (e.g., following this trend) for one more period, the patient may be at risk of medication overuse headache. The display can also include an instruction to seek medical advice 604. Additionally, the display can include an explanation of why there is an alert by explaining that in the two most recent periods the patient has had high use of pain/migraine medications (for at least 10 days each survey period).

In an additional embodiment, as illustrated in FIGS. 6A and 6B, the display on the graphical user interface 600 of the client device can include the dates of the first survey period 606a and the dates of the second survey period 606b and the type and amount of each medication type used during the respective survey period. Additionally, the display can include the number of days on which the patient experienced a headache or a migraine.

In an example embodiment, as illustrated in FIG. 6C, the display of the graphical user interface can include a summary with medication information 608. The medication information can include the different types of medication that the patient used during the survey period, the dosage of each medication, and the number of days the patient used the medication. As illustrated in FIGS. 6D and 6E the display can also include a listing of the different possible medications and the dosage of each medication similar to the display 400 in FIGS. 4A and 4B. The different medications can be listed under the “migraine” and “other” sub-menus and can include the dosages for each medication.

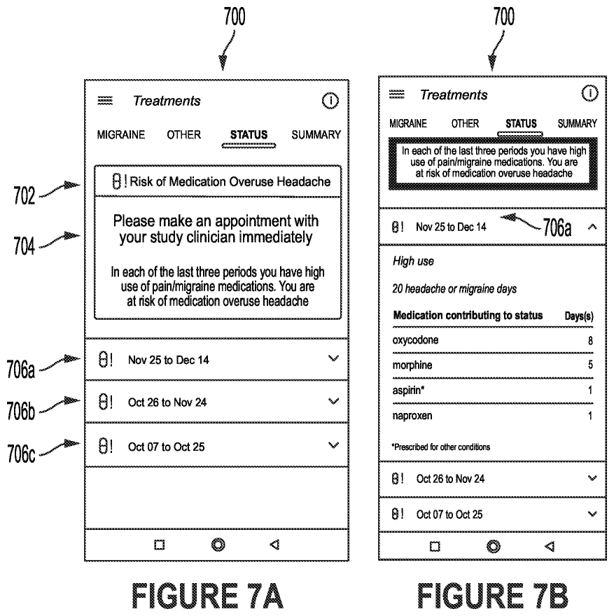

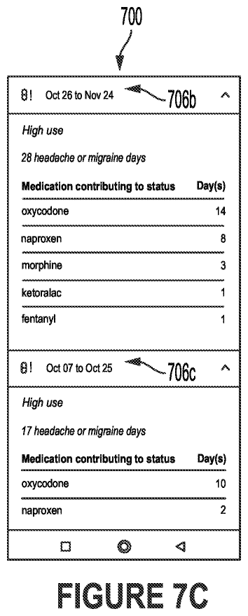

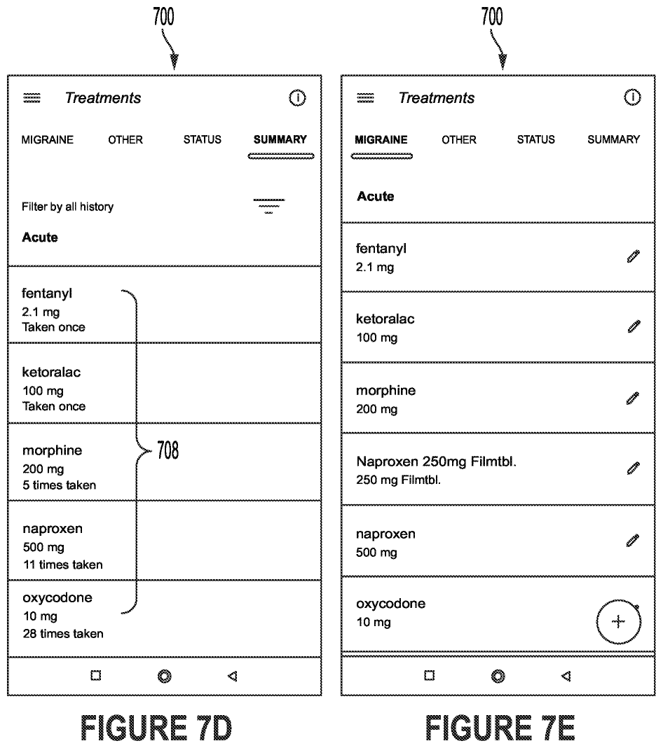

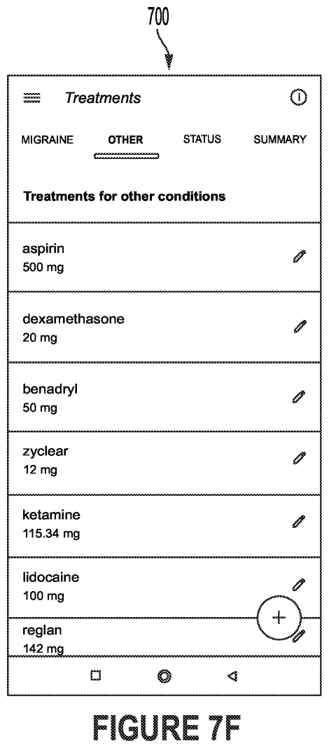

FIG. 7A illustrates an example embodiment of displaying the alert of medication overuse risk 702 via the graphical user interface 700 when the patient has overused medications for the most recent three survey periods in a row (i.e., for three months). As illustrated, the alert 702 on the graphical user interface 700 can include displaying a warning indication related to medication overuse 702 and an instruction to seek medical advice 704. Particularly, as illustrated in FIG. 7A, the warning can indicate that risk of medication overuse headache has been determined, and that the patient should make an appointment with their clinician immediately. Additionally, the display can include an explanation of why there is an alert by explaining that in each of the last three survey periods the patient has had high use of pain/migraine medication and the patient is at risk of medication overuse headache.

In an additional embodiment, as illustrated in FIGS. 7A-7C, the display on the graphical user interface 700 of the client device can include the dates of the first survey period 706a, the dates of the second survey period 706b, and the dates of the third survey period 706c. The display can also include the type and amount of each medication type used during the respective survey period. Additionally, the display can include the number of days on which the patient experienced a headache or a migraine.

In an example embodiment, as illustrated in FIG. 7D, the display of the graphical user interface can include a summary with medication information 708. The medication information can include the different types of medication that the patient used during the survey period, the dosage of each medication, and the number of days the patient used the medication. As illustrated in FIGS. 7D, 7E, and 7F the display can also include a listing of the different medications and the dosage of each medication similar to the display 400 in FIGS. 4A and 4B. The different medications can be listed under the “migraine” and “other” sub-menus and can include the dosages for each medication.

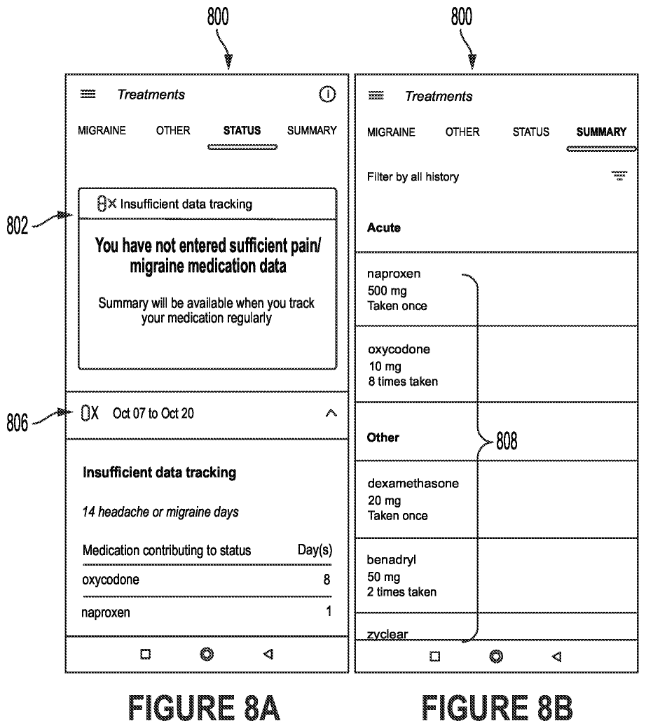

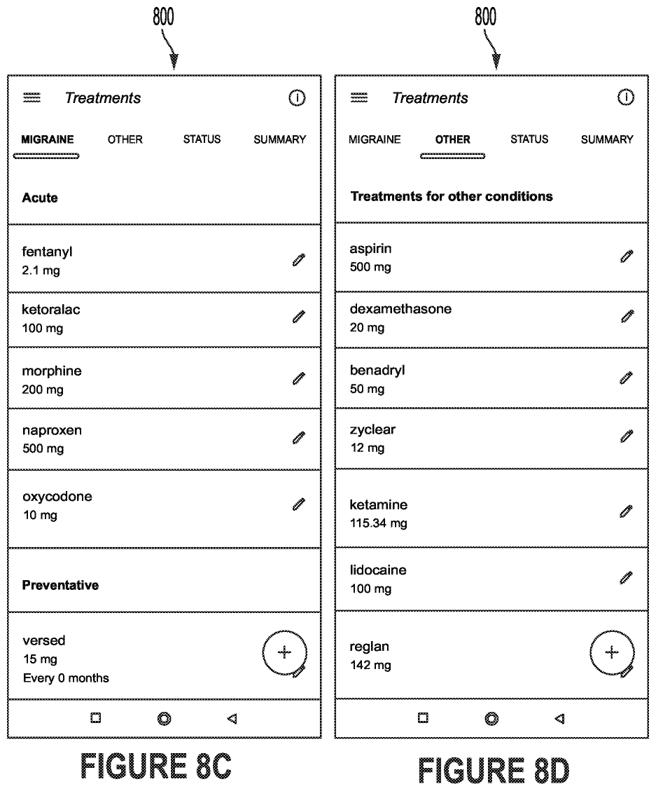

FIG. 8A illustrates an example embodiment of displaying an alert 802 informing the patient that there has not been enough data entered to track it and determine any trends. As illustrated, the alert 802 on the graphical user interface 800 can include displaying that the patient has not entered sufficient pain/migraine medication data (e.g., whether the patient has or has not taken medication) and that the summary may be available when the patient tracks medication regularly. In an additional embodiment, as illustrated in FIG. 8A, the display on the graphical user interface 800 of the client device can include the dates 806 that have been tracked thus far. The display can also include the type and amount of each medication type used during the respective survey period thus far. Additionally, the display can include the number of days on which the patient experienced a headache or a migraine.

In an example embodiment, as illustrated in FIG. 8B, the display of the graphical user interface can include a summary with medication information 808. The medication information can include the different types of medication that the patient used during the survey period thus far, the dosage of each medication, and the number of days the patient used the medication. As illustrated in FIGS. 8C and 8D the display can also include a listing of the different medications and the dosage of each medication similar to the display 400 in FIGS. 4A and 4B. The different medications can be listed under the “migraine” and “other” sub-menus and can include the dosages for each medication.

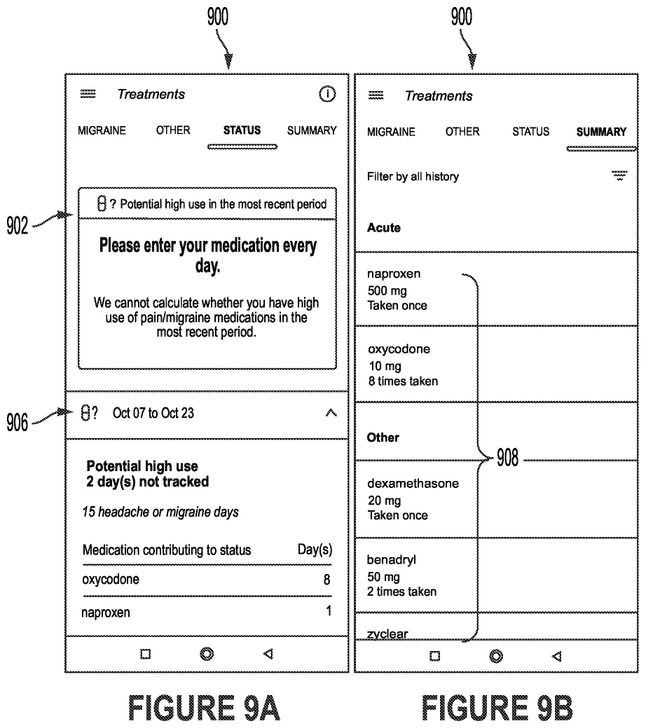

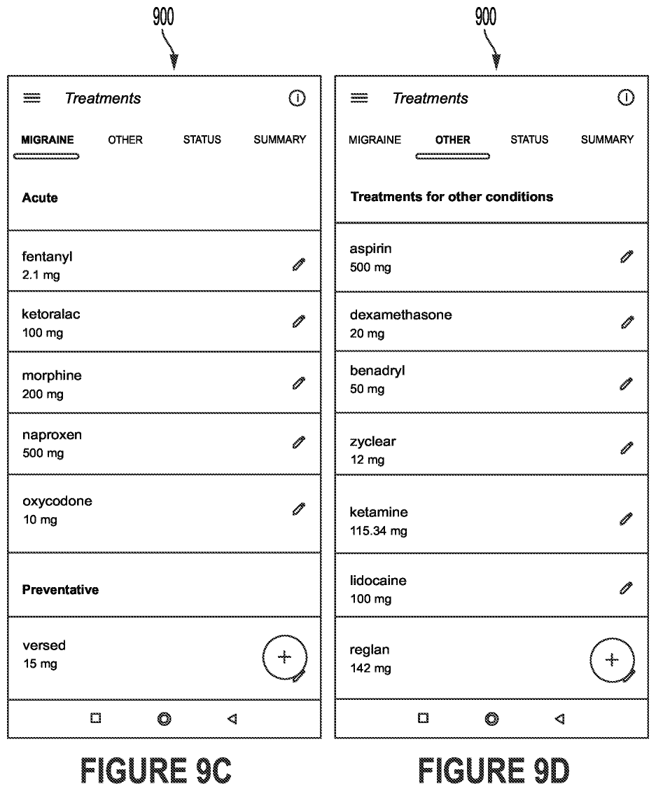

FIG. 9A illustrates an example embodiment of displaying an alert 902 informing the patient that there is potential high use in the most recent period. The alert of potential high use can be a result of the patient not tracking at least one day of medication use. When the patient misses a day, the method can include assuming the worst-case scenario. As illustrated, the alert 902 on the graphical user interface 900 can include displaying a reminder to enter medication every day and that the method cannot calculate whether the patient has high use of pain/medication medications in the most recent survey period. In an additional embodiment, as illustrated in FIG. 9A, the display on the graphical user interface 900 of the client device can include the dates 906 that have been tracked thus far. The display may also include a warning that there is potential high use and indicate if and how many days there is no medication data for. The display can also include the type and amount of each medication type used during the respective survey period thus far. Additionally, the display can include the number of days on which the patient experienced a headache or a migraine.

In an example embodiment, as illustrated in FIG. 9B, the display of the graphical user interface can include a summary with medication information 908. The medication information can include the different types of medication that the patient used during the survey period thus far, the dosage of each medication, and the number of days the patient used the medication. As illustrated in FIGS. 9C and 9D the display can also include a listing of the different medications and the dosage of each medication similar to the display 400 in FIGS. 4A and 4B. The different medications can be listed under the “migraine” and “other” sub-menus and can include the dosages for each medication.

The graphical user interface as previously described can also be used to schedule an appointment with a clinician. Via the graphical user interface, the patient can access a scheduling sub-menu through the menu. The sub-menu can display clinician availability via the graphical user interface for the client to select an appointment time with the clinician.

In an additional example embodiment displaying the alert of medication overuse risk includes displaying the alert of medication overuse risk on a personal medical avatar displayed via a graphical user interface. For example, the alert can appear at a text bubble or an icon on the personal medical avatar.

The alert of medication overuse risk can also be associated with a noise. For example, the client device can make a noise when there is an alert. The alert sound and volume can vary depending on the type of alert. For example, as the number of days that the medication is used increases, the volume of the alert can increase.

As previously discussed, the method 300 can include displaying the alert of medication overuse risk via the clinician dashboard including displaying the warning related to medication overuse and medication type being overused. When the trend is determined that indicates something related to medication overuse, an alert of medication overuse risk can be displayed on the clinician device belonging to a clinician via the clinician dashboard. The clinician can interact with the medication usage data and review the medication usage and other data via the clinician dashboard.

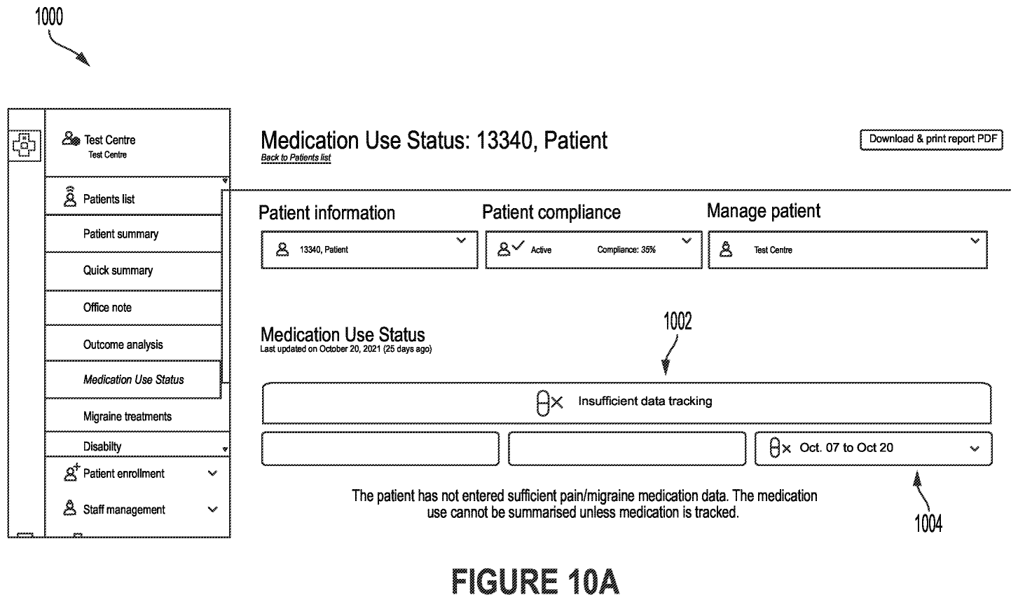

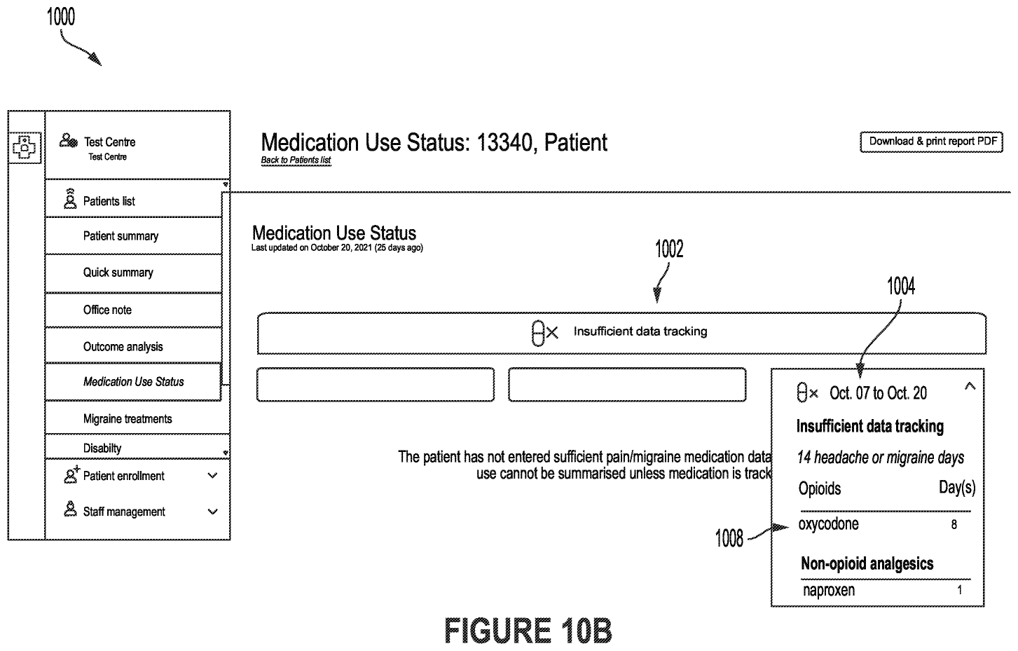

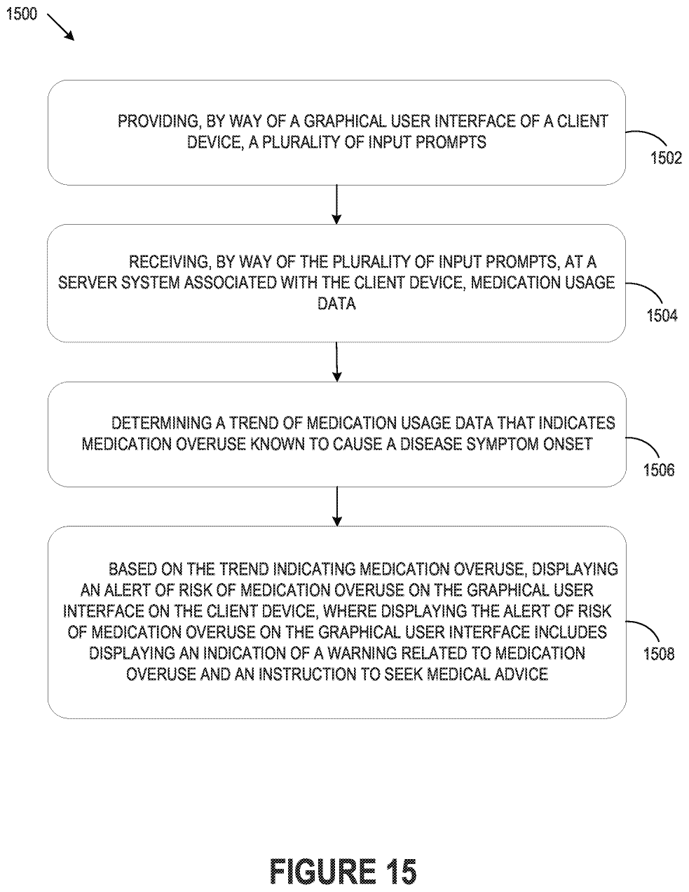

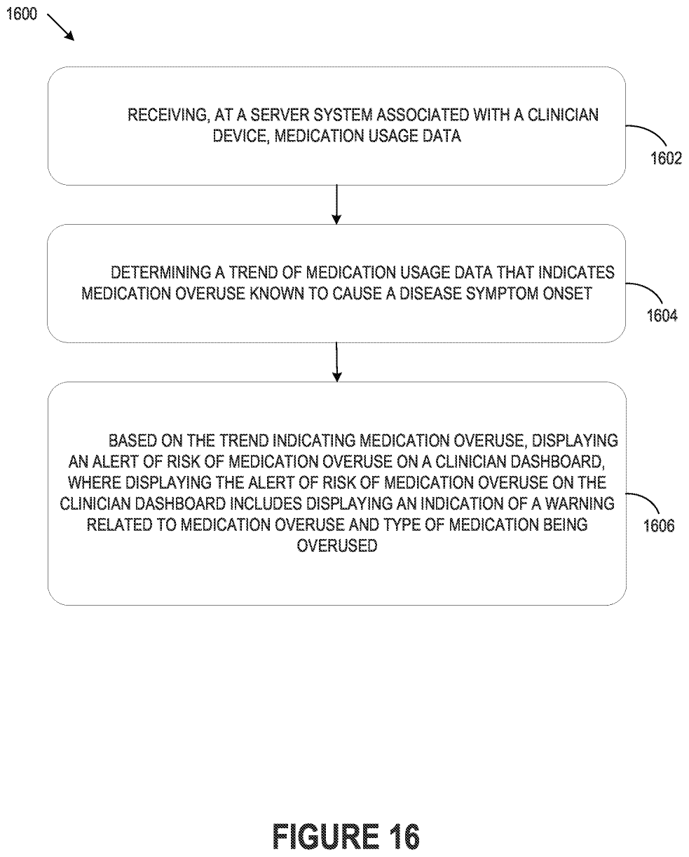

FIG. 10A-FIG. 18 illustrate an example of a clinician dashboard. The clinician dashboard can display information regarding patients and staff. The dashboard can include a menu where the clinician can select whether to view the patient list, patient enrollment, or staff management. Under each option there can be a sub-menu. Particularly, under the patients list the sub-menu can include options to view a patient summary, a quick summary, an office note, an outcome analysis, medication use status, migraine treatments, or disability. The display can also include which patient information is being displayed for, the patient's compliance in entering medication usage data, patient management data, and an indication of the last time the medication usage data was updated.

FIG. 10A illustrates an example embodiment of displaying an alert 1002 via the clinician dashboard 1000 informing the clinician that there has not been enough data entered to track and determine any trends. As illustrated, the alert 1002 on the clinician dashboard 1000 can include displaying that the patient has not entered sufficient pain/migraine medication data (e.g., whether the patient has or has not taken medication) so the medication use cannot be summarized. In an additional embodiment, as illustrated in FIG. 10A, the display on the clinician dashboard 1000 of the clinician device can include the dates 1004 that have been tracked thus far.

In an example embodiment, as illustrated in FIG. 10B, the display of the graphical user interface can include a summary with medication information 1008. The medication information displayed via the clinician dashboard can include the different types of medication in the medication usage data that the patient used during the survey period thus far, the dosage of each medication, and length of time each medication type was taken in the survey period (i.e., the number of days the patient used the medication). The display can further include the number of headache or migraine days the patient experienced.

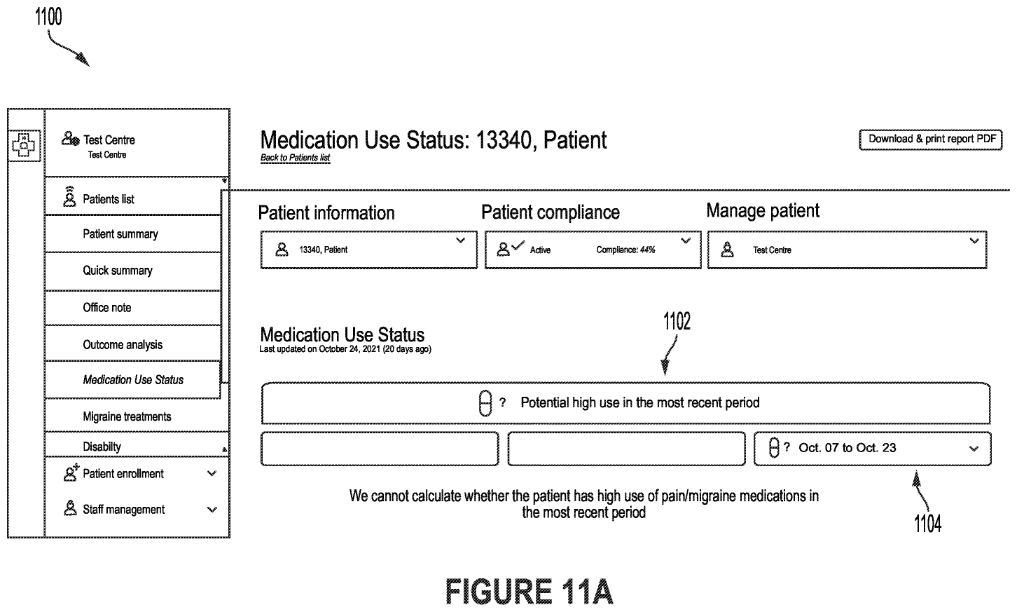

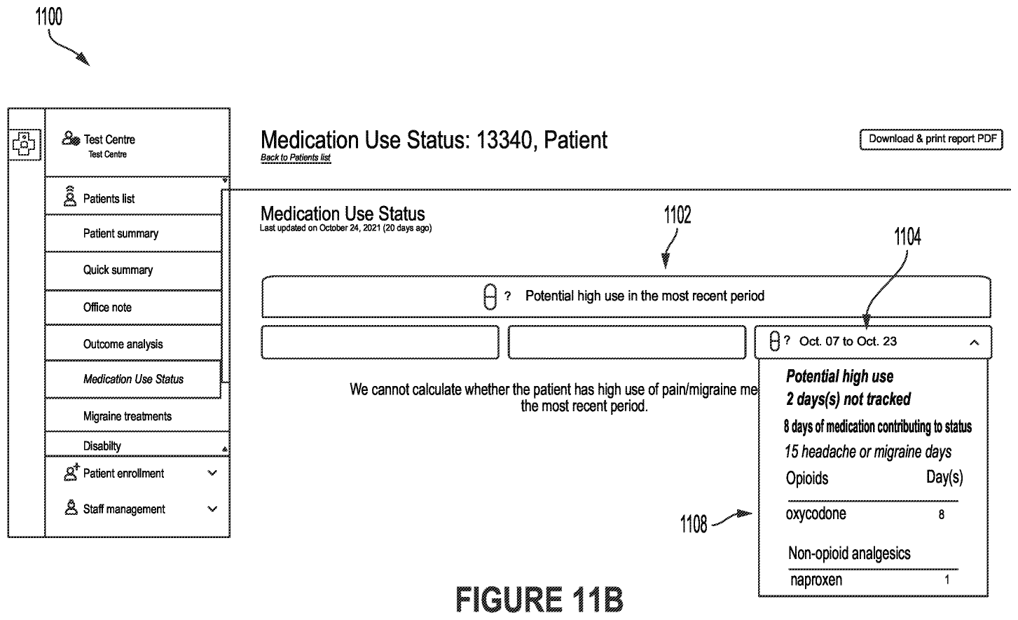

FIG. 11A illustrates an example embodiment of displaying an alert 1102 on the clinician dashboard 1100 informing the clinician that there is potential high use in the most recent survey period. The alert 1102 of potential high use can be a result of the patient not tracking at least one day of medication use. When the patient misses a day, the method can include assuming the worst-case scenario that the patient did take medication. As illustrated, the alert 1102 on the clinician dashboard 1100 can include displaying that whether the patient has high use of pain/migraine medication in the most recent survey period cannot be calculated. In an additional embodiment, as illustrated in FIG. 11A, the display on the clinician dashboard 1100 of the clinician device can include the dates 1104 that have been tracked thus far.

In an example embodiment, as illustrated in FIG. 11B, the display of the graphical user interface can include a summary with medication information 1108. The medication information displayed via the clinician dashboard can include the different types of medication in the medication usage data that the patient used during the survey period thus far, the dosage of each medication, length of time each medication type was taken in the survey period (i.e., the number of days the patient used the medication), and how many days in the survey period medication use was not tracked for. The display can further include the number of headache or migraine days the patient experienced.

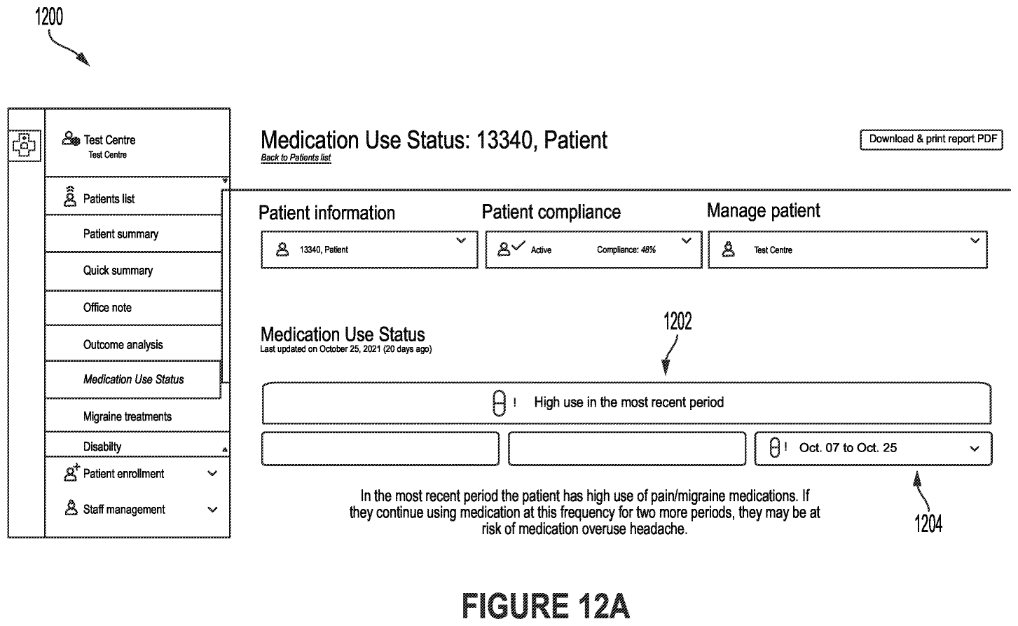

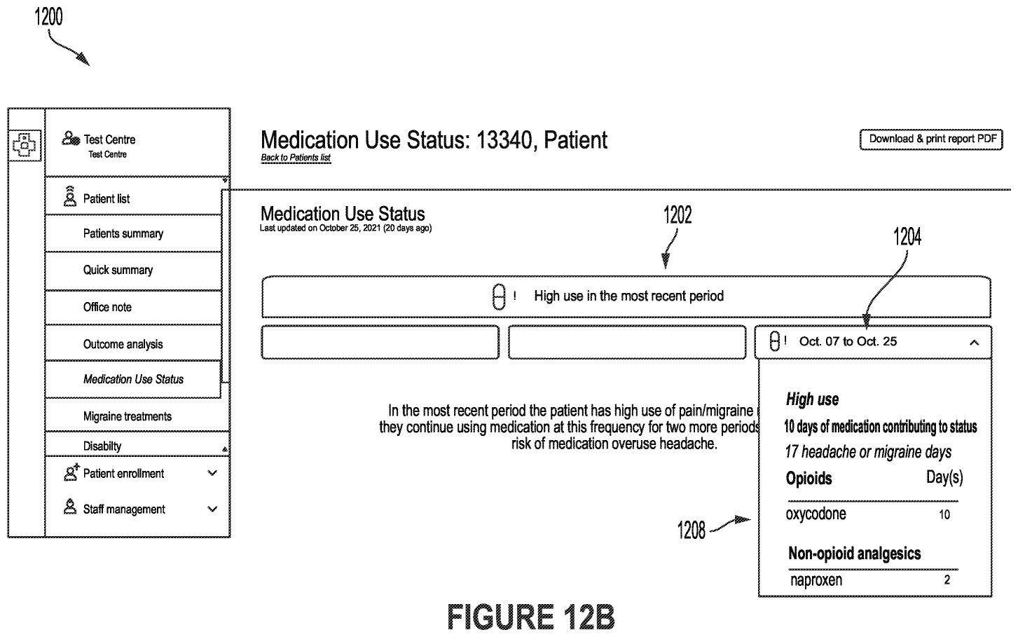

FIG. 12A illustrates an example embodiment of displaying an alert 1202 via the clinician dashboard 1200 informing the clinician that there is high use in the most recent survey period. The alert 1202 of high use can be a result of the patient overusing medication in the most recent period. As illustrated, the alert 1202 on the clinician dashboard 1200 can include displaying that in the most recent survey period the patient has high use of pain/migraine medication and that if the patient continues using medication at this trend for two more periods the patient is at risk of medication overuse headache. In an additional embodiment, as illustrated in FIG. 12A, the display on the clinician dashboard 1200 of the clinician device can include the dates 1204 that have been tracked thus far.

In an example embodiment, as illustrated in FIG. 12B, the display of the graphical user interface can include a summary with medication information 1208. The medication information displayed via the clinician dashboard can include the different types of medication in the medication usage data that the patient used during the survey period thus far, the dosage of each medication, and length of time each medication type was taken in the survey period (i.e., the number of days the patient used the medication). The display can further include the number of headache or migraine days the patient experienced.

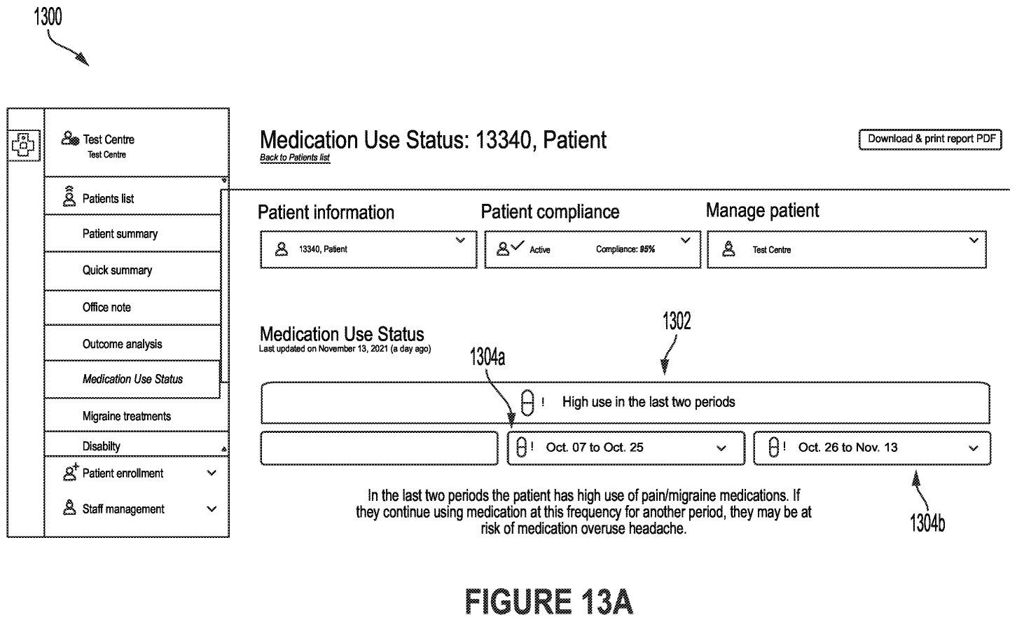

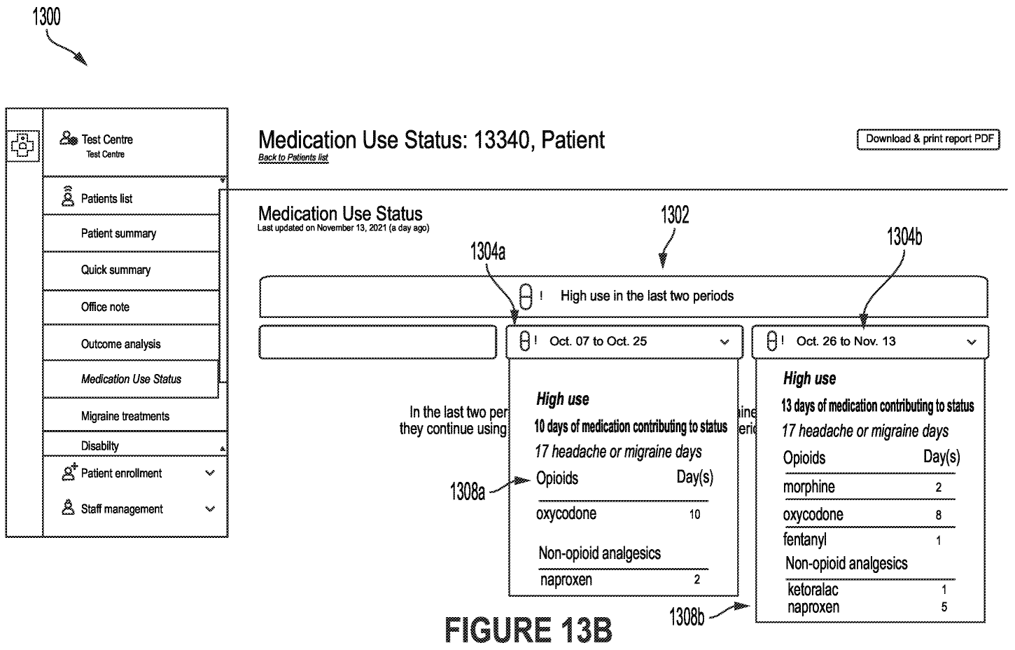

FIG. 13A illustrates an example embodiment of displaying an alert 1302 via the clinician dashboard 1300 informing the clinician that there is high use in the two most recent survey periods. The alert 1302 of high use can be a result of the patient overusing medication in the two most recent periods. As illustrated, the alert 1302 on the clinician dashboard 1300 can include displaying that in the two most recent survey periods the patient has high use of pain/migraine medication and that if the patient continues using medication at this trend for one more survey period the patient is at risk of medication overuse headache. In an additional embodiment, as illustrated in FIG. 13A, the display on the clinician dashboard 1300 of the clinician device can include the dates of the first survey period 1304a and the dates of the second survey period 1304b.

In an example embodiment, as illustrated in FIG. 13B, the display of the graphical user interface can include a summary with medication information related toward the first survey period 1308a and medication information related toward the second survey period 1308b. The medication information for each survey period displayed on the clinician dashboard can include the different types of medication in the medication usage data that the patient used during the respective survey period, the dosage of each medication, and length of time each medication type was taken in the respective survey period (i.e., the number of days the patient used the medication). The display can further include the number of headache or migraine days the patient experienced in each survey period.

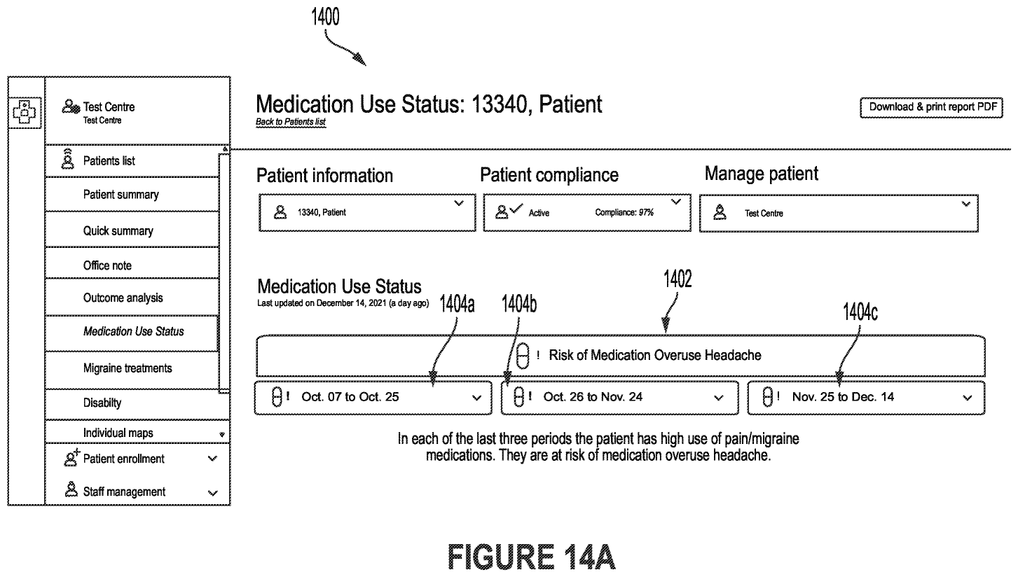

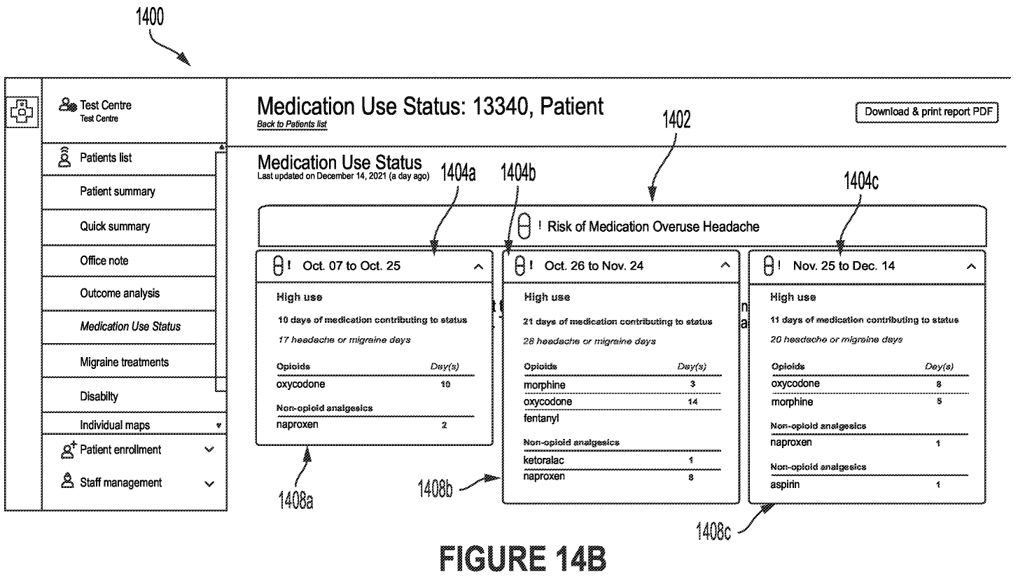

FIG. 14A illustrates an example embodiment of displaying an alert 1402 via the clinician dashboard 1400 informing the clinician that there is risk of medication overuse headache. The alert 1402 of risk of medication overuse headache can be a result of the patient overusing medication in the three most recent periods. As illustrated, the alert 1402 on the clinician dashboard 1400 can include displaying that in the three most recent survey periods the patient has high use of pain/migraine medication, and the patient is at risk of medication overuse headache. In an additional embodiment, as illustrated in FIG. 14A, the display on the clinician dashboard 1400 of the clinician device can include the dates of the first survey period 1404a, the dates of the second survey period 1404b, and the dates of the third survey period 1404c.

In an example embodiment, as illustrated in FIG. 14B, the display of the graphical user interface can include a summary with medication information related toward the first survey period 1408a, medication information related toward the second survey period 1408b, and medication information related toward the third survey period 1408c. The medication information for each survey period displayed via the clinician dashboard can include the different types of medication in the medication usage data that the patient used during the respective survey period, the dosage of each medication, and length of time each medication type was taken in the respective survey period (i.e., the number of days the patient used the medication) The display can further include the number of headache or migraine days the patient experienced in each survey period.

In an additional example embodiment displaying the alert of medication overuse risk includes displaying the alert of medication overuse risk on a personal medical avatar displayed via the clinician dashboard. For example, the alert can appear at a text bubble or an icon on the personal medical avatar.

The alert of medication overuse risk can also be associated with a noise. For example, the clinician device can make a noise when there is an alert. The alert sound and volume can vary depending on the type of alert. For example, as the number of days that the medication is used increases, the volume of the alert can increase.

An embodiment can further include scheduling an appointment for the patient with the clinician. It can be desirable for the patient to meet with the clinician in order to discuss the medications that the patient is taking to preempt the occurrence of medication overuse and medication overuse headache. In one example embodiment the method can include requesting the clinician's availability through the clinician dashboard in response to determining the trend of medication usage data indicates medication overuse. The clinician can provide their availability in response to the request and an appointment can be scheduled. The patient appointment can then be displayed via the graphical user interface on the client device. In an additional embodiment, the clinician can be provided, by way of the clinician dashboard, a scheduling prompt. The clinician can approve the prompt and a request to schedule an appointment can be sent to the patient via the server system. The server system can receive a plurality of inputs including the scheduling request. The server can then send the scheduling request to the graphical user interface of the client device. The scheduling request can include an instruction to schedule an appointment with a clinician displayed via the graphical user interface of the client device. The patient can then schedule an appointment with the clinician via the graphical user interface.

Method 1500 is a flow chart of a method of displaying an alert of medication overuse risk on the client device. As previously described, medication overuse headache risk can occur when the patient has overused a medication for at least three months. Any of the previously described methods can be applied to method 1500. Method 1500 may include one or more operations, functions, or actions, as depicted by one or more of blocks 1502, 1504, 1506, and 1508 each of which may be carried out by any of the systems shown in prior figures, among other possible systems.

Those skilled in the art will understand that the flow charts described herein illustrate functionality and operation of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art.

At block 1502, method 1500 involves providing a plurality of input prompts via a graphical user interface of a client device. In an example embodiment, the input prompts can include a request for data relating to days with intake of symptomatic headache medications, at least one medication type, medication dosage, medication administration route, and total number of medication doses taken in a day, days with headache, type of headache, and severity of headache.

At block 1504, method 1500 includes receiving medication usage data via the plurality of input prompts, at a server system associated with the client device. In an example embodiment, the medication usage data includes days with intake of symptomatic headache medications, at least one medication type, medication dosage, medication administration route, and/or total number of medication doses taken in a day. In one embodiment, the medication type and medication dosage can be scanned from a barcode and input into the graphical user interface. The information gathered from the barcode can then be sent to the server system to assist in determining whether there is a trend indicating medication overuse.

At block 1506, method 1500 includes determining a trend of medication usage data that indicates medication overuse known to cause a disease symptom onset. The trend can be determined as previously described relating to block 306.

At block 1508, method 1500 includes displaying an alert of medication overuse risk via the graphical user interface on the client device based on the trend indicating medication overuse. Displaying the alert of medication overuse risk via the graphical user interface includes displaying a warning indication related to medication overuse and an instruction to seek medical advice. The display can be similar to the display in FIGS. 4A through 9D.

In an example embodiment, once the trend indicating a medication overuse risk has been determined, the graphical user interface can display an instruction to reduce or eliminate medication use until contacting a clinician. This instruction can assist in preventing the onset of medication overuse headache.

An embodiment can further include scheduling an appointment for the patient with the clinician. It can be desirable for the patient to meet with the clinician in order to discuss the medications that the patient is taking to preempt the occurrence of medication overuse and medication overuse headache. In one example embodiment, an instruction to schedule an appointment with a clinician in response to the trend of medication usage data indicating medication overuse can be displayed the graphical user interface on the client device.

The patient can also use the graphical user interface to enter other instructions. For example, the patient can limit the number of alerts they receive. Via the settings of the graphical user interface, the patient can enter an instruction to limit the frequency of alerts to a predetermined threshold. The graphical user interface can receive the instruction to limit the frequency of alerts to the predetermined threshold, and reduce the frequency of alerts displayed via the graphical user interface to the predetermined threshold.

In an example embodiment, the patient can also view a patient report. The patient report can be a complete breakdown of all of their medication usage data. The patient report can include a plurality of survey periods during which medication was tracked, types of medication taken during a respective survey period, number of days a respective medication was taken, and a classification of medication overuse risk based on the plurality of survey periods. The patient report is illustrated in FIGS. 5A, 5B, 6A-6C, 7A-7D, 8A, 8B, 9A, and 9B.

Method 1600 is a flow chart of a method of displaying an alert of medication overuse risk via the clinician dashboard of the clinician device. As previously described, medication overuse headache can occur when the patient has overused a medication for at least three months. Any of the previously described methods can be applied to method 1600. Method 1600 may include one or more operations, functions, or actions, as depicted by one or more of blocks 1602, 1604, and 1606 each of which may be carried out by any of the systems shown in prior figures, among other possible systems.

Those skilled in the art will understand that the flow charts described herein illustrate functionality and operation of certain implementations of the present disclosure. In this regard, each block of the flowchart may represent a module, a segment, or a portion of program code, which includes one or more instructions executable by one or more processors for implementing specific logical functions or steps in the process. The program code may be stored on any type of computer readable medium, for example, such as a storage device including a disk or hard drive.

In addition, each block may represent circuitry that is wired to perform the specific logical functions in the process. Alternative implementations are included within the scope of the example implementations of the present application in which functions may be executed out of order from that shown or discussed, including substantially concurrent or in reverse order, depending on the functionality involved, as would be understood by those reasonably skilled in the art.

At block 1602, method 1600 includes receiving, at a server system associated with a clinician device, medication usage data. In an example embodiment, the medication usage data includes days with headache, headache characteristics on each day with headache, days with intake of symptomatic headache medications, at least one medication type, medication dosage, medication administration route, and total number of medication doses taken in a day. In an example embodiment, certain medications can be disregarded and are not considered when determining the trend. The server system can receive, from the clinician device via the clinician dashboard, a request to disregard at least one medication type when determining the trend of medication usage data, and disregard the at least one medication type determining the trend of medication usage data.

An example embodiment can further include, determining a number of days with headache are over a predetermined headache threshold over a survey period. The number of days with headache can be based on the data stored in the medication usage data. Determining a number of days with headache are over a predetermined headache threshold over a survey period can include that (i) the number of days with headache over the survey period is greater than a previous number of days over a previous survey period, and/or (ii) at least one headache characteristic from over the survey period is different from headache characteristics from over the previous survey period. The method can further include determining daily intake of symptomatic headache medications is over a threshold of recommended medication usage days over the survey period. Based on the determinations, the method can include displaying an alert of medication overuse headache risk via the clinician dashboard. The alert can include displaying an indication that daily intake of symptomatic headache medications over the survey period is over a threshold of recommended medication usage days and at least one of: (i) the number of days with headache over the survey period is greater than a previous number of days over a previous survey period, and/or (ii) at least one headache characteristic from over the survey period is different from headache characteristics from over the previous survey period.

At block 1604, method 1600 includes determining a trend of medication usage data that indicates medication overuse known to cause a disease symptom onset. The trend can be determined as previously described relating to block 306.

At block 1606, method 1600 includes displaying an alert of medication overuse risk on a clinician dashboard on the clinician device based on the trend indicating medication overuse. Displaying the alert of medication overuse risk via the clinician dashboard includes displaying a warning indication related to medication overuse and medication type being overused. The display can be similar to the display in FIGS. 10A through 14B.

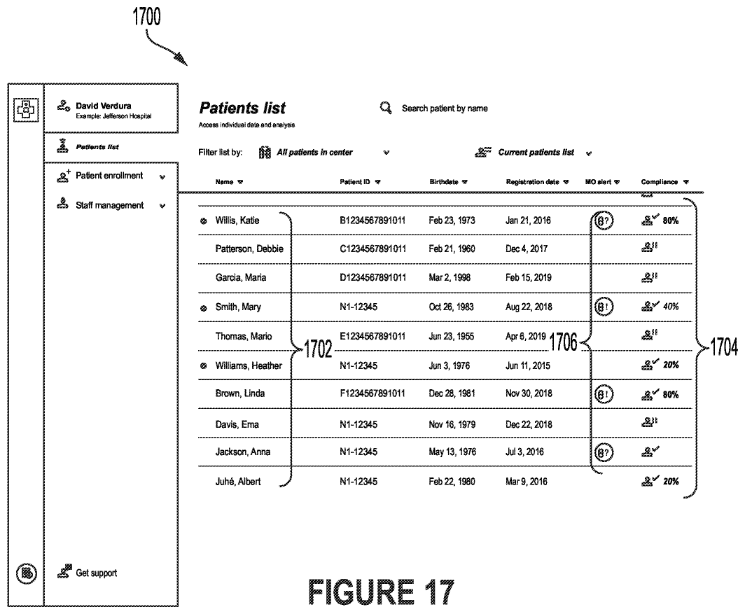

FIG. 17 illustrates a patient list 1700 display of the clinician dashboard. In an example embodiment, the clinician dashboard can display a list of patients 1702, a level of compliance 1704, and a level of medication overuse headache risk for each patient 1706. The list of patients can include all of the patients that are under the clinician's care. By selecting a patient, the clinician can view all of the medication usage data associated with the selected patient.

An embodiment can further include scheduling an appointment between the patient and the clinician. It can be desirable for the patient to meet with the clinician in order to discuss the medications that the patient is taking to preempt the occurrence of medication overuse and medication overuse headache. In one example embodiment, the clinician dashboard can display, a request to schedule an appointment for a patient in response to determining the trend of medication usage data indicating medication overuse. When the request is displayed, the clinician can enter availability and send it back to the patient to make an appointment. Alternatively, the clinician can immediately schedule the appointment.

In an example embodiment, the methods included herein can also be used to assist with diagnosis of medication overuse headache based on the guidelines set forth in the ICHD. The method can include (i) comparing headache characteristics on each day with headache over a survey period and with headaches that occurred before and after the period, (ii) determining a difference in headache characteristics over the survey period and a difference between headache characteristics over the survey period and after the survey period. (iii) determining daily intake of symptomatic headache medications is over a threshold of recommended medication usage days over the survey period, and (iv) displaying an alert of medication overuse headache risk via the clinician dashboard. Displaying the alert of medication overuse headache risk via the clinician dashboard can include displaying an indication of the difference in headache characteristics over the survey period and if the daily intake of symptomatic headache medications is over the threshold of recommended medication usage days. Indicating that the headache characteristics have changed, and that medication use is over the threshold can assist the clinician in diagnosing medication overuse headache.

The method can further include (i) determining days in the survey period with headaches, (ii) determining days in the survey period with intake of symptomatic headache medications, (iii) determining an overlap in the days in the survey period with headaches and the days in the survey period with intake of symptomatic headache medications, and (iv) displaying an alert of medication overuse headache risk on a clinician dashboard. Displaying the alert of medication overuse headache risk via the clinician dashboard can include displaying an indication of the overlap in the days in the survey period with headaches and the days in the survey period with intake of symptomatic headache medications. By highlighting the overlap, the method can assist a doctor in diagnosing medication overuse headache.

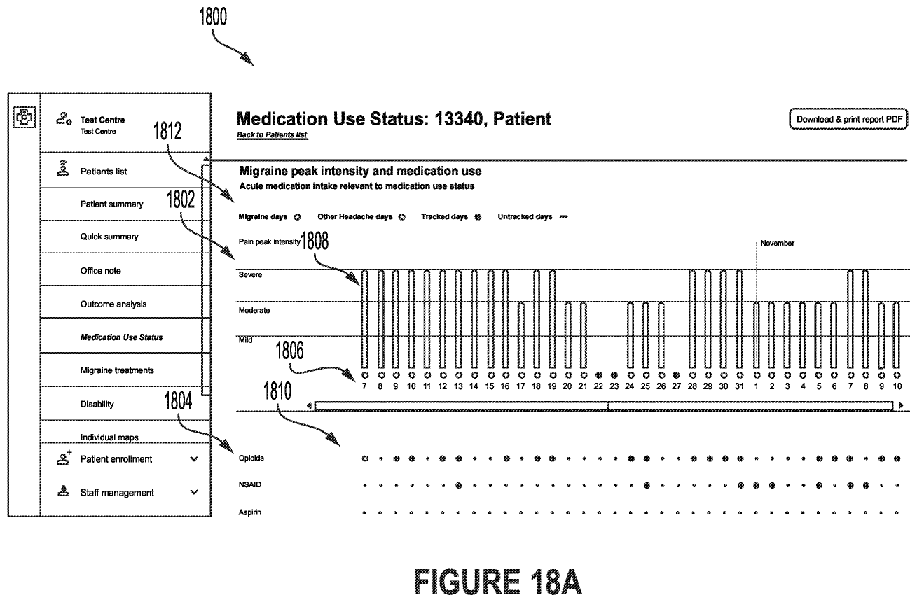

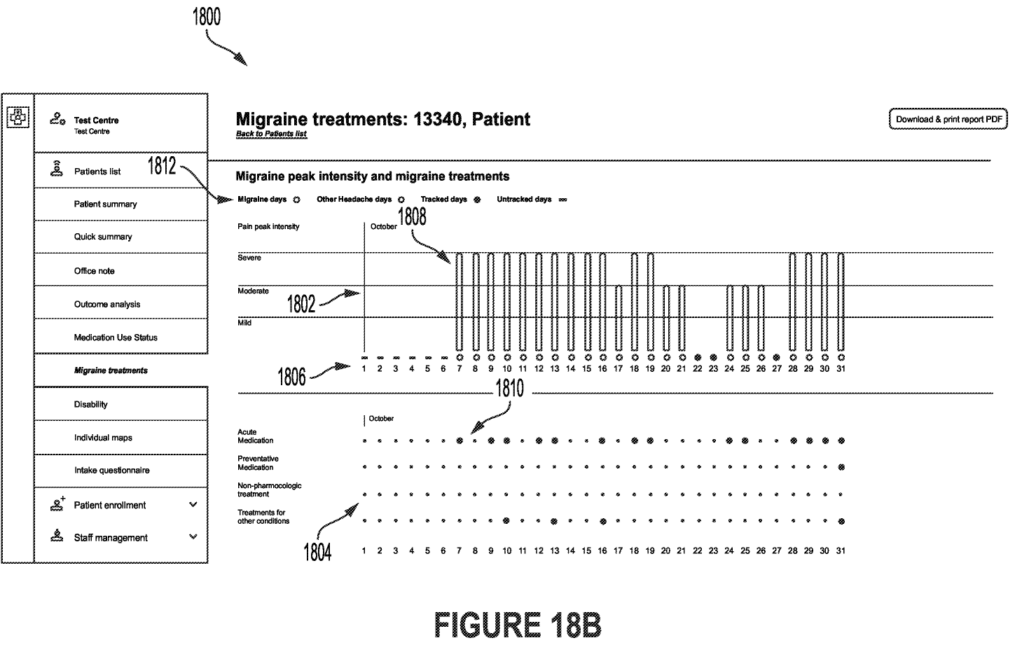

In an example embodiment, the method can include displaying a chart 1800 to illustrate the overlap. FIG. 18A illustrates the chart 1800 for displaying the indication of the overlap in the days in the survey period. The chart 1800 can include a first vertical axis 1802, a second vertical axis 1804, and a horizontal axis 1806. The horizontal axis 1806 can include an indication of days in the survey period. The days can be identified by month and day of the month. The first vertical axis 1802 can include an indication of pain intensity laid out as mild, moderate, or severe. The second vertical axis 1804 can include an indication of types of medication. For example, in FIG. 18 the types of medication are opioids, NSAID, and aspirin. The chart 1800 can include a first icon 1808 and a second icon 1810. The first icon can represent pain intensity and headache type on days in the survey period. For example, the pain intensity can be represented as a bar graph while the type of headache can be represented by dots with different colors and fills. The second icon can represent types of medication taken on days in the survey period. The second icon can be represented by dots next to the types of medications. The display can further include a key 1812 to explain the different icons. FIG. 18B illustrates a similar chart with different types of medications.

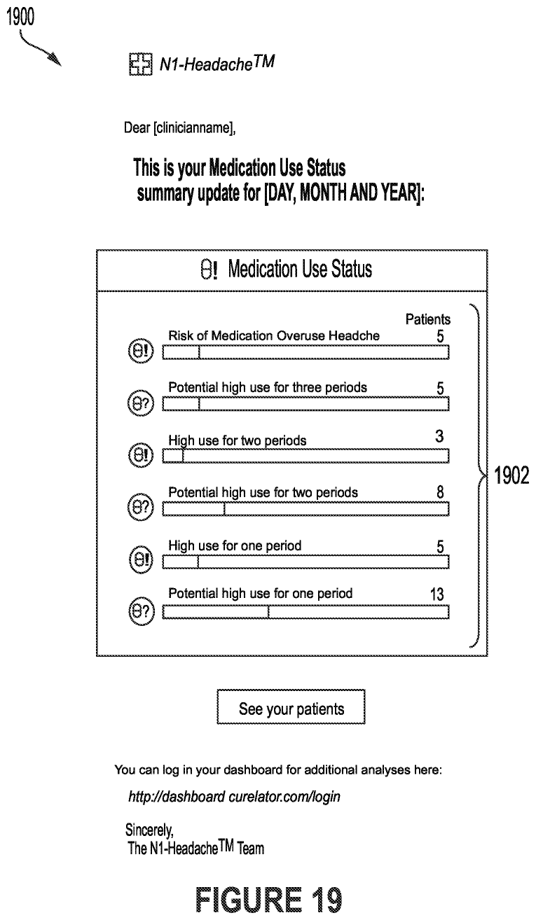

Example embodiments can also include sending periodic updates to a clinician detailing the status of each patient. FIG. 19 illustrates a medication use status summary 1900 for a plurality of patients. The summary is broken down by how many patients fall into a plurality of categories 1902. The categories include medication overuse headache risk, potential high use for three periods, high use for two periods, potential high use for two periods, high use for one period, and potential high use for one period.

The systems and methods described herein generally relate to software application technology, and more particularly to healthcare management software. Software applications are typically most effective where users remain engaged and when chum rates are low. Similarly, healthcare management applications rely on consistent data from individuals with a patient population to arrive at meaningful conclusions. Accordingly, it is desirable within the software application and healthcare industry to promote user/patient engagement. One way of promoting user engagement is by providing beneficial recommendations based on data provided by the user in a visually appealing way. The above-described examples achieve this by receiving patient data, then generating and displaying an alert related to medication use. This manner of providing a visual aid that evolves similar to the patient establishes a direct relationship between user engagement and the alerts. In this way, a healthcare management application “rewards” users for engagement, and thus encourages continued use of the software. Accordingly, the described embodiments further drives user engagement by providing visualizations that are personalized for each patient. Thus, the systems and methods described herein provide specific implementations directed towards improving the technical fields of (i) software applications and (ii) healthcare management platforms.

For similar reasons, the systems and methods described herein effectuate improvements in computing systems associated with healthcare management. For example, the determined method may be used to store large amounts of medication usage data, predict, and diagnose medication overuse headache before it occurs. In this manner, computing requirements of the system can be improved by solving the problem of diagnosing patients. These and other improvements to relevant technical fields will be understood to those having skill in the art.

While particular aspects and embodiments are disclosed herein, other aspects and embodiments will be apparent to those skilled in the art in view of the foregoing teaching. For example, while the embodiments and examples are described with respect to migraine headaches, the disclosed systems and methods are not so limited and can be applicable to a broad range of disease symptoms and related disease factors and disease triggers. The various aspects and embodiments disclosed herein are for illustration purposes only and are not intended to be limiting, with the true scope being indicated by the following claims.

What is claimed is:

1. A method comprising:
providing a plurality of input prompts via a graphical user interface on a client device;
receiving medication usage data via the plurality of input prompts at a server system associated with at least one of the client device and a clinician device;
determining a trend of medication usage data that indicates medication overuse known to cause a disease symptom onset; and
based on the trend indicating medication overuse, displaying an alert of medication overuse risk via at least one of the graphical user interface on the client device and a clinician dashboard on the clinician device,
wherein displaying the alert of medication overuse risk via the graphical user interface comprises displaying a warning indication related to medication overuse and an instruction to seek medical advice, and wherein displaying the alert of medication overuse risk via the clinician dashboard comprises displaying the warning related to medication overuse and medication type being overused.

2. The method of claim 1, further comprising:
requesting clinician availability through the clinician dashboard in response to determining the trend of medication usage data indicating medication overuse;
scheduling a patient appointment; and
displaying the patient appointment via the graphical user interface on the client device.

3. The method of claim 1, further comprising:
providing a scheduling prompt via the clinician dashboard;
receiving a plurality of inputs comprising a scheduling request, at the server system; and
displaying an instruction to schedule an appointment with a clinician via the graphical user interface of the client device.

4. The method of claim 1, further comprising receiving the medication usage data via the plurality of input prompts over a survey period, wherein the trend of medication usage data that indicates medication overuse known to cause disease symptom onset is determined over the survey period.

5. The method of claim 4, further comprising displaying, via the clinician dashboard, types of medication in the medication usage data and length of time each medication type was taken in the survey period.

6. The method of claim 4, wherein determining medication usage data that indicates a trend of medication overuse known to cause disease symptom onset comprises:
receiving a usage of a medication over the survey period at the server system associated with the client device; and
determining daily usage of the medication is over a threshold of recommended medication usage days over the survey period.

7. The method of claim 6, wherein the survey period comprises one month, and wherein the warning related to medication overuse further comprises a warning that the trend of medication overuse may indicate a risk of disease symptom onset within two months.

8. The method of claim 6, wherein the survey period comprises two months, and wherein the warning related to medication overuse further comprises a warning that the trend of medication overuse may indicate a risk of disease symptom onset within one month.

9. The method of claim 6, wherein the survey period comprises three months, and wherein the warning related to medication overuse further comprises a warning that the trend of medication overuse may indicate a risk of disease symptom onset.

10. The method of claim 6, further comprising:
determining at least one missing day of medication usage from the daily usage over the survey period; and
predicting daily usage for the at least one missing day of medication usage using a trained machine learning model,
wherein determining the trend of medication usage data that indicates medication overuse known to cause disease symptom onset is further based on the predicted daily usage.

11. The method of claim 1, wherein the medication usage data comprise days with intake of symptomatic headache medications, at least one medication type, medication dosage, medication administration route, and total number of medication doses taken in a day.

12. The method of claim 11, wherein the medication type comprises at least one of prescribed medication and non-prescribed medication.

13. The method of claim 11, further comprising:
receiving, at the server system, from the clinician device via the clinician dashboard, a request to disregard the at least one medication type when determining the trend of medication usage data; and
disregarding the at least one medication type while determining the trend of medication usage data.

14. The method of claim 1 wherein displaying the alert of medication overuse risk via at least one of the graphical user interface on the client device and the clinician dashboard on the clinician device comprises displaying the alert of medication overuse risk on a personal medical avatar.

15. A method comprising:
providing a plurality of input prompts via a graphical user interface of a client device;
receiving medication usage data via the plurality of input prompts at a server system associated with the client device;
determining a trend of medication usage data that indicates medication overuse known to cause a disease symptom onset; and
based on the trend indicating medication overuse, displaying an alert of medication overuse risk via the graphical user interface on the client device,
wherein displaying the alert of medication overuse risk via the graphical user interface comprises displaying a warning indication related to medication overuse and an instruction to seek medical advice.

16. The method of claim 15, further comprising displaying, via the graphical user interface on the client device, an instruction to schedule an appointment with a clinician in response to the trend of medication usage data indicating medication overuse.

17. The method of claim 15, further comprising displaying, via the graphical user interface, an instruction to reduce or eliminate medication use until contacting a clinician.

18. The method of claim 15, further comprising:
receiving an instruction to limit alert frequency to a predetermined threshold via the graphical user interface of the client device; and
reducing the frequency of alerts displayed via the graphical user interface to the predetermined threshold.

19. The method of claim 15, wherein the medication usage data comprise days with intake of symptomatic headache medications, at least one medication type, medication dosage, medication administration route, and total number of medication doses taken in a day.

20. The method of claim 19, wherein the medication type comprises at least one of prescribed medication and non-prescribed medication.

21. The method of claim 19, wherein the medication type and medication dosage are scanned from a barcode.

22. The method of claim 15, wherein the plurality of input prompts further comprises a plurality of disease symptom inputs and wherein the plurality of inputs further comprises disease symptom data and wherein the disease symptom data comprises days with headache and headache characteristics on each day with headache.

23. The method of claim 15, further comprising displaying a patient report, wherein the patient report comprises a plurality of survey periods, types of medication taken during a respective survey period, number of days a respective medication was taken, and a classification of medication overuse risk based on the plurality of survey periods.

24. A method comprising:
receiving medication usage data at a server system associated with a clinician device;
determining a trend of medication usage data that indicates medication overuse known to cause a disease symptom onset; and
based on the trend indicating medication overuse, displaying an alert of medication overuse risk via a clinician dashboard based on the trend indicating medication overuse,
wherein displaying the alert of medication overuse risk via the clinician dashboard comprises displaying a warning indication related to medication overuse and medication type being overused.

25. The method of claim 24, further comprising displaying, via the clinician dashboard, a request to schedule an appointment for a patient in response to determining the trend of medication usage data indicating medication overuse.

26. The method of claim 24, wherein the medication usage data comprises days with headache, headache characteristics on each day with headache, days with intake of symptomatic headache medications, at least one medication type, medication dosage, medication administration route, and total number of medication doses taken in a day.

27. The method of claim 26, further comprising:
determining a number of days with headache are over a predetermined headache threshold over a survey period wherein at least one of: (i) the number of days with headache over the survey period is greater than a previous number of days over a previous survey period, and (ii) at least one headache characteristic from over the survey period is different from headache characteristics from over the previous survey period;
determining daily intake of symptomatic headache medications is over a threshold of recommended medication usage days over the survey period; and
displaying an alert of medication overuse headache risk via the clinician dashboard that comprises displaying an indication that daily intake of symptomatic headache medications over the survey period is over a threshold of recommended medication usage days and at least one of: (i) the number of days with headache over the survey period is greater than a previous number of days over a previous survey period, and (ii) at least one headache characteristic from over the survey period is different from headache characteristics from over the previous survey period.

28. The method of claim 26, further comprising:
comparing headache characteristics on each day with headache over a survey period;
determining a difference in headache characteristics over the survey period;
determining daily intake of symptomatic headache medications is over a threshold of recommended medication usage days over the survey period; and
displaying an alert of medication overuse headache risk via the clinician dashboard that comprises displaying an indication of the difference in headache characteristics over the survey period and the daily intake of symptomatic headache medications is over the threshold of recommended medication usage days.

29. The method of claim 28, further comprising:
determining days in the survey period with headaches;
determining days in the survey period with intake of symptomatic headache medications;
determining an overlap in the days in the survey period with headaches and the days in the survey period with intake of symptomatic headache medications; and
displaying the alert of medication overuse headache risk via the clinician dashboard by displaying an indication of the overlap in the days in the survey period with headaches and the days in the survey period with intake of symptomatic headache medications.

30. The method of claim 29, wherein displaying an indication of the overlap in the days in the survey period comprises:
displaying a chart, wherein the chart comprises a first vertical axis, a second vertical axis, and a horizontal axis;
wherein the horizontal axis comprises an indication of days in the survey period;
wherein the first vertical axis comprises an indication of pain intensity;
wherein the second vertical axis comprises an indication of types of medication; and
a first icon, wherein the first icon represents pain intensity and headache type on days in the survey period, and a second icon, wherein the second icon represents types of medication taken on days in the survey period.

31. The method of claim 24, further comprising:
receiving, at the server system, from the clinician device via the clinician dashboard, a request to disregard at least one medication type when determining the trend of medication usage data; and
disregarding the at least one medication type determining the trend of medication usage data.

32. The method of claim 24, further comprising displaying, via the clinician dashboard, a list of patients and a level of medication overuse risk for each patient.
